(19) United States
(12) Patent Application Publication
(10) Pub. No.: US 2026/0014067 A1
(43) Pub. Date: Jan. 15, 2026

(54) ENZYME BASED COMPOSITIONS AND METHODS FOR REMOVING DENTAL CALCULI

(71) Applicant: PONTIS BIOLOGICS, INC., Huntington, NY (US)

(72) Inventor: Dennis C. Mynarcik, Huntington, NY (US)

(21) Appl. No.: 19/145,152

(22) PCT Filed: Jan. 5, 2024
(86) PCT No.: PCT/US2024/010512

§ 371 Date: Jul. 1, 2025

Related U.S. Application Data
(60) Provisional application No. 63/478,641, filed on Jan. 5, 2023.

(51) Int. Cl. A61K 8/66; A61K 8/25; A61K 8/34; A61K 8/60; A61K 8/73; A61K 8/86; A61Q 11/00; C12N 9/22; C12N 9/76

(52) U.S. Cl. CPC A61K 8/66; A61K 8/25; A61K 8/345; A61K 8/60; A61K 8/73; A61K 8/86; A61Q 11/00; C12N 9/22; C12N 9/6427; A61K 2800/87; C12Y 301/21001; C12Y 304/21001

(57) ABSTRACT

Disclosed are compositions and formulations for removing dental calculus which comprise enzymes or other biocatalysts which cleave (i) surface-accessible nucleic acid polymers and (ii) glycoprotein carbohydrate chains at galactose residues. Optionally, one or more proteolytic enzymes are included. The compositions destroy the structural integrity of the calculus, allowing it to be readily removed without requiring special treatment by a trained dental professional. Also disclosed are methods for removing dental calculus using the disclosed compositions and formulations.

### CROSS REFERENCE TO RELATED APPLICATIONS

The present application claims the benefit of U.S. Provisional Patent Application No. 63/478,641, entitled, “ENZYME BASED COMPOSITIONS AND METHODS FOR REMOVING DENTAL CALCULI” filed Jan. 5, 2023, the contents of which is hereby incorporated by reference in its entirety.

### FIELD

The present disclosure relates to compositions and methods for reducing and removing dental plaque and calculus in human and non-human animals.

### INCORPORATION OF SEQUENCE LISTING

The present application contains a Sequence Listing which has been submitted in .XML format via Patent Center and is hereby incorporated by reference in its entirety. Said WIPO Sequence Listing was created on Jan. 4, 2024, named “119879-783212 Sequence Listing.xml”, and is 8 kilobytes in size.

### BACKGROUND

Dental tartar, also referred to as dental calculus, is a fossilized/mineralized substance that, if not removed, progressively accumulates and ultimately leads to periodontal diseases such as gingivitis and periodontitis resulting in a chronic inflammatory state that can predispose to diabetes or heart disease in humans and non-human animals, including but not limited to dogs and cats. Presently, methods of removing calculus are performed by trained dental professionals at best once or twice a year and are essentially limited to scraping of the tooth surface using metal alloy dental picks or an ultrasonic device to fragment and dislodge the calculus from tooth enamel. Calculus is not effectively removed by dentifrice formulations such as toothpastes and mouth rinses used in routine daily oral hygiene practice.

The World Health Organization estimates oral diseases affect nearly 3.5 billion people globally with untreated dental caries being the most common health condition, and 1 billion people having severe periodontal diseases. The estimated number of cases of oral diseases globally is about 1 billion higher than mental disorders, cardiovascular disease, diabetes mellitus, chronic respiratory diseases and cancers combined. Moreover, it is the most prevalent health problem in high income nations. In the United States, data from the 2018 NHANES estimated that 42% of dentate US adults have periodontitis and over 25% of adults in the United States have untreated tooth decay. However, oral health diseases exert systemic manifestations beyond the oral cavity such as diabetes, cardiovascular disease and pre-term babies with low birth weights. The linkage is not precisely defined, but commonly associated with a chronically inflammatory state, originating with periodontal inflammation. Oral biofilms are the main etiologic factor for a variety of oral diseases such as dental caries, and periodontal diseases. An integral feature of biofilms, including dental plaque, is extracellular DNA (eDNA) that aids in the formation of the gel nature of plaque. Bacteria living in dental biofilms are the primary etiological factors of periodontal diseases. In addition, bacterial species living in dental biofilm also metabolize sugar and produce acids that reduce the biofilm fluid pH. This leads to a mineral discrepancy between acidic biofilm fluid and tooth, resulting in loss of tooth mineral composition, clinically noted as loss of tooth structure and cavitations. The control and prevention of dental biofilms, plaque, and dental calculus accumulations are the goal of a healthy oral environment.

Oral prophylaxis, especially tooth brushing, is a basic and daily custom for almost all people, even in developing countries, and its aim is to remove dental plaque. However, more than 40% of plaque will not be removed, even by a well-trained person, and may become mineralized to become dental calculus. Current toothpastes are not very effective at preventing dental calculus formation but rather decrease the rate at which calculus forms. For example, a Cochrane Collaboration publication stated after six months of use, triclosan/copolymer toothpaste participants had a mean total calculus of 12.49 mm, and the control group was 14.61 mm. Both groups formed measurable amounts of dental calculus though the triclosan/copolymer toothpaste group had 15% less dental calculus formed and therefore slows but does not prevent the formation of dental calculus.

Thus, a crucial unmet need exists for a product for regular home use that will reduce, remove, and/or prevent dental calculus.

### SUMMARY

Among the various aspects of the present disclosure are a first dental composition comprising or consisting essentially of at least two calculus targeting enzymes. Other aspects and features of the disclosure are detailed below.

In one aspect the current disclosure encompasses a dental composition comprising a DNase, chymotrypsin or a functional variant or derivative thereof, and an orally acceptable additive, carrier or excipient or any combination thereof. In some aspects, the dental composition is a dentifrice.

In some aspects, the dental composition may not include β-galactosidase.

In some aspects, the dental composition may include DNAse in an amount of about 50,000 to about 250,000, or about 100,000 to about 200,000, or about 150,000 to about 200,000, or 150,000, or 160,000, or 170,000, or 180,000, or 185,000 or 190,000, or 200,000 Kunitz units/mL.

In some aspects, the dental composition may also include a DNAse, wherein the DNAse is DNAse I or a functional variant or derivative thereof.

In some aspects, the dental composition may also include chymotrypsin or a functional variant or derivative thereof wherein the chymotrypsin or a functional variant or derivative is present in an amount of about 100 to about 5,000, or about 500 to about 4,000, or about 1000 to about 3000, or 1,500, or 1,600, or 1,700, or 1,800, or 1,900, or 2,000, or 2,100, or 2,200, or 2,300, or 2,400, or 2,500, or 2,600, or 2,700, or 2,800, or 2,900 or 3,000 units/mL.

In some aspects, the dental composition may also include an orally acceptable additive where the orally acceptable additive is a thickener and/or a gelling agent selected from gellan gum (low acyl or high acyl), glycerol, silica, guar gum, xanthan gum, polyethylene glycols, polyvinyl pyrrolidones and co-polymers thereof, polylactic acids, polyglyocolic acids, long chain fatty acid alcohols, cellulose-based polymers, acrylate polymers and any combination thereof. In some aspects, the dental composition is a dentifrice.

In some aspects, the dental composition may also include orally acceptable carriers for example ethanol, isopropanol, glycerol, sorbitol or any combination thereof.

In some aspects, the dental composition may further include a surfactant for example a ceteareth or a steareth.

In some aspects, the dental composition includes DNase 1 or a functional variant or derivative thereof, chymotrypsin or a functional variant or derivative thereof, and one or more of, 10-20% glycerol, 0.00001-0.000001% Ceteareth-25, 0.1%-0.5% Xanthan gum, 1-10% sorbitol, and 5-25% silica. The composition may further include water.

In some aspects, the dental composition is in the form of a paste or gel, for example a toothpaste or dentifrice, or toothpowder, or mouth wash. In some aspects, the dental composition is a dentifrice. In some aspects, the dental composition is combined with a dental tray, a capsule applicator, an oral pick or a dental floss to provide an oral hygiene device.

In some aspects, the current disclosure also encompasses use of the dental composition or device comprising the dental composition, for removing calculus dentalis in a subject in need thereof. In some aspects, dental composition is used as a dentifrice. In some aspects, the subject is a mammal. In some aspects, the subject is a human.

In one aspect, the present disclosure provides a method for reducing or removing calculus dentalis in a subject in need thereof. The method includes contacting a tooth surface with an effective amount of a dental composition which includes DNAse I or a functional variant or derivatives thereof, and chymotrypsin or a functional variant or derivative thereof. In some aspects, dental composition is formulated as a dentifrice.

In some aspects, the dental composition does not comprise β-galactosidase.

In some aspects, the composition further includes an orally acceptable additive, carrier or excipient or a combination thereof.

In some aspects, the composition includes a paste or a liquid and contacting a tooth surface includes application of the composition using a brush, a tray, by oral rinse or any combination thereof. In some aspects, dental composition is formulated as a dentifrice.

The method may also include where the method includes at least once daily, at least twice daily or at least three-times daily applications for a period of at least a day or more, a week or more, a month or more or a year or more.

In some aspects, the disclosure provides a method for reducing or removing calculus dentalis in a subject in need thereof, where the method includes contacting a tooth surface with an effective amount of dental composition for example a dentifrice.

In some aspects, the subject is a mammal. In some aspects the subject is a canine, equine, feline or primate. In some aspects, the subject is a human.

In some aspects the current disclosure also encompasses a kit, wherein the kit includes the dental composition and instructions for use. In some aspects, the kit comprises the dental composition as a dentifrice. The kit may further include at least one applicator selected from a toothbrush, an oral pick, a dental floss, and a dental tray.

Other technical features may be readily apparent to one skilled in the art from the following figures, descriptions, and claims.

### BRIEF DESCRIPTION OF THE DRAWINGS

Aspects of the present disclosure are illustrated in the following figures:

FIG. 1 is a schematic of the proof-of-concept in vivo study design. Randomized trial to evaluate the safety and efficacy of the exemplary formulations to remove existing calculus deposits over a 4-week trial period.

FIG. 2 provides an overview of the scores associated with the Volpe-Manhold Index. Scores are based on cumulative calculus score, sum of lingual (side toward tongue) surfaces of the 6 lower anterior (Front) teeth.

FIG. 3 shows that the formulation significantly reduced total tartar as estimated by Volpe-Manhold scores.

FIG. 4 shows the % change in tartar across all the subjects of the trial. Change in tartar was consistent across all subjects; all but 2 control patients had increased in tartar and all, but one subject showed reduced tartar.

FIG. 5 shows the positive reaction of subjects on the disclosed formulation.

FIG. 6 depicts the overview of daily brushing schedule by study arm.

FIG. 7 displays the individual V-MI values at baseline and at 4 weeks for the different treatment groups.

FIG. 8 shows percent of patients who gave a favorable score to how clean their teeth felt (“4” or “5” on a 5-point scale). Score was rated on a 5-point scale where “1” is the least clean you could imagine and “5” is the cleanest you could imagine.

### DETAILED DESCRIPTION

The present disclosure provides new dental compositions and formulations for reducing or removing dental calculus, methods of their use to reduce or remove dental calculus, and kits comprising the compositions and formulations in humans and non-human animals, including but not limited to dogs and cats.

### Definitions

Unless defined otherwise, all technical and scientific terms used herein have the meaning commonly understood by a person skilled in the art to which this disclosure pertains. The following references provide one of skill with a general definition of many of the terms used in this disclosure: Singleton et al., Dictionary of Microbiology and Molecular Biology (2nd Ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “optional” or “optionally” mean that the subsequently described event or circumstance may or may not occur, and that the description includes instances where said event or circumstance occurs and instances where it does not.

As used herein, the terms “calculus” or “tartar” are used interchangeably and refers to a form of hardened dental plaque. It is caused by precipitation of minerals from saliva and gingival crevicular fluid in plaque on the teeth. This process of precipitation kills the bacterial cells within dental plaque, but the rough and hardened surface that is formed provides an ideal surface for further plaque formation. This leads to calculus buildup, which can compromise the health of the gingiva (gums). Calculus can form both along the gumline, where it is referred to as supragingival, and within the narrow sulcus that exists between the teeth and the gingiva, where it is referred to as subgingival.

The term “calculus targeting enzyme” as used herein refers to any one of: (i) an enzyme that hydrolyze(s) the sugar-phosphate ester linkages of deoxyribonucleic acid (DNA) or ribonucleic acid (RNA); (ii) a proteolytic enzyme that hydrolyzes amino acid linkages in protein backbone and optionally (iii) an enzyme that hydrolyzes glycoprotein carbohydrate at linkages containing galactose, N-acetyl-glucosamine, fucose, N-acetyl-galactosamine, or sialic acid. Calculus targeting enzymes include but are not limited to a DNase, Chymotrypsin and other proteases, including but not limited to specific enzymes as disclosed elsewhere herein. In various aspects, the calculus targeting enzyme is not beta-galactosidase, i.e., a composition as disclosed herein may specifically exclude beta-galactosidase.

The terms “polypeptide” and “protein,” as used interchangeably herein, refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation or any other manipulation or modification, such as conjugation with a labeling component. Also included within the definition are, for example, polypeptides containing one or more analogs of an amino acid (including, for example, unnatural amino acids, etc.), as well as other modifications known in the art. For example, as used herein the protein or polypeptide sequence of an enzyme (by way of non-limiting example, DNaseI (also referred to as DNAse 1), or Chymotrypsin) also encompasses modifications as provided herein, derivatives and variants of the polypeptide.

The terms “derivative” or “variant,” when used herein with reference to a polypeptide, refers to a polypeptide related to a wild-type polypeptide, for example either by amino acid sequence, structure (e.g., secondary and/or tertiary), activity (e.g., enzymatic activity) and/or function. Derivatives, or variants of a polypeptide can comprise one or more amino acid variations (e.g., mutations, insertions, and deletions), truncations, modifications, or combinations thereof compared to a wild-type polypeptide. The term “functional variant or derivative” as used herein encompasses a protein that functions at the same level or has enhanced or reduced functionality as compared to its wild-type polypeptide.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used herein, “and/or” should be understood as specifically disclosing each of the two specified features or components with, or without the other. For example, “A and/or B” is to be taken as specific disclosure of each of (i) A, (ii) B and (iii) A and B, the same as if each were set out individually herein.

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combinations.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification.

These are only examples of what is specifically intended, and all possible combinations of numerical values between and including the lowest value and the highest value enumerated are to be considered to be expressly stated in this disclosure.

As used herein, “individual”, “subject”, “host”, and “patient” can be used interchangeably herein and refer to any mammalian subject for whom diagnosis, treatment, prophylaxis or therapy is desired, for example, humans, pets, livestock, horses or other animals. As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, dog, cat, and the like. In some aspects, the subject can be a human.

As used herein, the terms “administer,” “administering,” or “administration,”, refer to orally administering a compound or composition, regardless of form. For example, the methods disclosed herein include administration of an effective amount of a compound or composition to achieve the desired or stated effect. The compositions disclosed herein can optionally be administered to the oral cavity or specific portions of the oral cavity (for e.g., one or more teeth, or one or more portions of one or more teeth (e.g., exposed enamel, dentine, or cementum)) using one or more devices known in the art, e.g., brushes (e.g., toothbrushes), floss or flossers (e.g., dental floss), irrigators (e.g., oral irrigators), picks (e.g., dental picks or toothpicks), scrapers (e.g., tongue cleaners or scrapers), spatulas, sticks, retainers dental tray, or mouth guards, etc.

As used herein, the terms “treat”, “treating,” or “treatment,”, refer to partially or completely alleviating, inhibiting, ameliorating, or relieving the disease or condition from which the subject is suffering. This means any manner in which one or more of the symptoms of a disease or disorder (for e.g., calculus) can be ameliorated or otherwise beneficially altered. As used herein, amelioration of the symptoms of a particular disorder (for e.g., calculus) refers to any lessening, whether permanent or temporary, lasting or transient that can be attributed to or associated with treatment by the compositions and methods of the present disclosure. In some aspects, treatment can promote or result in, for example, a decrease in amount of calculus (for e.g., in a subject) relative to the amount of calculus prior to treatment; or reductions in one or more symptoms associated with one or more periodontal diseases (e.g., gingivitis) in a subject relative to the subject's symptoms prior to treatment.

As used herein terms “prevent,” “preventing,” and “prevention,” refer to a decrease in the occurrence of a disease or decrease in the risk of acquiring a disease or its associated symptoms in a subject. The prevention may be complete, e.g., the total absence of disease in a subject. The prevention may also be partial, such that the occurrence of the disease in a subject is less than, occurs later than, or develops more slowly than that which would have occurred without the present disclosure.

As various changes could be made in the above-described cells and methods without departing from the contemplated scope of the disclosure, it is intended that all matter contained in the above description and in the examples given below, shall be interpreted as illustrative and not in a limiting sense.

### (I) Dental Calculus

Calculus has a complex composition and will vary between different individuals, dependent on diet, genetics (subtle compositional variations in saliva) and the makeup of the oral microbiome, but the mechanism of its deposition remains to be determined. In a recent report (Aghanashini, S., et al., J. of Health Sciences and Res. (2016) 7:42-50) six theories of calculus formation were reviewed: the Booster Mechanism; the Epitactic concept; the Inhibition theory; the Transformation theory; the Bacterial Theory; and the Enzymatic theory. All but the Bacterial theory focus on inorganic components. The Bacterial theory hypothesizes that bacteria attaching to the tooth surface is responsible for calculus formation. Studies from the United States in the 1960s and 1970s focused on the composition of calculus hydrolysates and did not venture to identify the macromolecular organic components, if any. Since then, further progress in the U.S. has not been forthcoming. Thus, the field has not consolidated around a single view of calculus development.

Recent studies of dental calculus harvested from ancient teeth have assessed for the presence of DNA and demonstrated that DNA is an extremely rugged molecule. DNA is negatively charged and, in addition to DNA, calculus contains salivary glycoproteins, mucin being the most abundant member that is negatively charged, as well. Calcium is positively charged and abundant in saliva mediating salt bridges between the negative charges in DNA and Mucins. An additional component of calculus formation is the protein meshwork created by the activity of transglutaminase, which crosslinks glutamine residues on one protein with a lysine residue on an adjacent protein molecule. This process is thought to originate at the tooth gingival interface, where surface proteins on the epithelial surface become crosslinked to soluble salivary proteins creating a meshwork that extends from the epithelium to the enamel surface. Thus, at the tooth/gingival interface, proteins on the epithelial surfaces become cross-linked to other salivary and food proteins by the enzyme transglutaminase, creating an additional foundational material for mineralization.

The model informing the present disclosure begins with chemically clean tooth enamel in the normal oral environment. Without intending to be bound by theory, it is believed that DNA and mucin will bind to positively charged calcium ions in the outer surface of the enamel hydroxyapatite (Ca₁₀(PO₄)₆(OH)₂), and that salivary calcium will further bind to the outer face of the enamel-associated DNA and mucins, initiating a laminated arrangement by attracting more calcium, DNA and mucins. It is further believed that the less abundant but much larger DNA from the oral environment will associate with available hydroxyapatite calcium and with the calcium bound to the outer face of the immobilized mucins. DNA and mucin can be approximated as cylindrical structures with the charged groups arrayed radially. Therefore, only a fraction of the charged groups will occupy a face of the molecule that is interacting with the underlying matrix (¼-5 in the case of DNA). Thus, the effectiveness of cooperative interactions relies upon the molecular length to supply the strength needed to make the interaction of sufficient duration in a highly hydrated environment. By reducing the length of the DNA polymer or cleaving the protein and/or carbohydrate chains, terminating in negative charges, from the glycoprotein peptide backbone, the strength of the cooperative interactions is reduced. With progressive reduction of the negative polymer length by cleavage of DNA and increased cleavage of glycoprotein chains from glycoprotein peptide backbones, the cooperativity integral to calculus and plaque structure is lost, allowing the constituents to be washed away.

DNA is recognized as highly resistant to cleavage. Chemical hydrolysis of DNA requires boiling it in acid. The relationship of the length of DNA, relative to a bacterial cell, is on a macro-scale, but the role of DNA and glycoproteins like mucin in the formation of plaque and calculus is on the molecular scale. The number of associative events that mucin and DNA can achieve are enormous, contributing to the strength of the calculus aggregate. Thus, DNA's extensive length and chemical imperviousness makes it a strategic component of calculus. While a glycoprotein is not as rugged as DNA, it is sufficiently sturdy to play an important structural role. Both glycoprotein and DNA's association with immobilized calcium is enhanced through cooperativity. In this context, cooperativity is like the base pairing of double stranded DNA. While the DNA base pair interactions are established by hydrogen bonding (weak, compared with ionic salt bridges that will dominate in calculus) the precise geometry of the double helix, and the large number of base pairs makes separating the two strands difficult enough to require temperatures approaching boiling. In the case of calculus formation, when one salt bridge comes undone, through competition with water, those remaining upstream and downstream salt bridges will retain the two undone bridge partners in close enough proximity to facilitate their rapid re-association.

A purely chemical approach to the removal of calculus would require a chemical environment that would be chemically extreme, resulting in damage to the oral cavity. However, a biologic/enzymatic approach would be a non-toxic, effective and an efficient approach to disintegrating dental calculus. DNA can be cleaved by the enzyme deoxyribonuclease (DNase) anywhere along its length, at accessible sugar-phosphate bonds, reducing the length of the DNA polymer to short stretches of DNA (oligonucleotides) with the proportional loss of cooperativity. The element of the mucin glycoprotein that forms the associations with calcium are the sulfate and sialic acid groups (both negatively charged) at the ends of the carbohydrate chains. The carbohydrate chains of salivary mucin are diverse and large. They terminate in neutral sugars (56%), sialic acid (26%) and sulfate (19%) and vary in average chain lengths of 13 units, 17 units and 41 units, respectively (Thomsson, K. A., Glycobiology (2002) 12: 1-14). The carbohydrate chains are composed predominantly of galactose, fucose, and N-acetylglucosamine with lesser quantities of N-acetyl galactose. The mucin glycoproteins dimerize end-to-end and then go on to form higher-order structures. The microbes of the oral and gut microbiome have evolved to exploit the mucin carbohydrates as a nutrient source. They secrete N-acetyl-glucosaminidase, beta-galactosidase, N-acetyl-galactosidase, fucosidase, neuraminidase (sialidase) to cleave the carbohydrate chains into smaller sizes for nutrient uptake (Derrien, M., Gut Microbes (2010) 1:254-268). While the most accessible region to cleave the carbohydrate chain is in the middle of the chain at the galactose and N-acetyl-glucosamine residues with the enzymes beta-D-galactosidase and beta-N-acetyl-D-glucosaminidase, respectively, any cleavage that removes the acidic sulfates and sialic acid units from the protein chain will eliminate the cooperativity, important in the calculus and plaque architecture.

While the DNA/mucin/calcium complexes are forming, they are mixed with other salivary proteins and proteins from the diet that are continuously crosslinked by transglutaminase. With time more calcium diffuses into the aggregate, further compacting the calculus as it mineralizes. This emphasizes the role for an additional enzyme that targets the polypeptide component of these glycoproteins (for example, chymotrypsin) was also found herein to be effective in enhancing breakdown of calculus and plaque architecture.

Calculus is a solid, thus it exposes only a 2-dimensional surface to the environment. Therefore, a single enzyme, such as a DNAse activity, is limited to digesting only the DNA that is surface accessible. It is unable to access the DNA that is buried beneath the exposed layer of cross-linked protein. The same holds true if only a protease, such as a chymotryptic activity is used. It digests the surface-exposed crosslinked protein meshwork until at remains surface-exposed is exclusively a salt-bridged DNA/mucin layer, impenetrable to a protease. This necessitates using enzymes in combination to progressively attack the calculus surface.

### (II) Dental Compositions and Formulations

Compositions and formulations according to the present disclosure comprise at least two calculus targeting enzymes and may include a combination of two or more calculus targeting enzymes each with different catalytic activities that hydrolyze different chemical constituents in the structure of calculus. Calculus targeting enzymes include enzymes that hydrolyze any one of: (i) the sugar-phosphate ester linkages of deoxyribonucleic acid (DNA) or ribonucleic acid (RNA); (ii) proteins, including glycoproteins; and (iii) glycoprotein carbohydrates. The calculus targeting enzymes, both DNases, RNases and protease enzymes, act upon the nucleic acids and glycoprotein elements that are readily accessible at the surface of dental plaque and calculus. As the surface-accessible DNA is cleaved by DNase into shorter segments, for example, the shorter segments will have lost their capacity for cooperative associations with the underlying matrix, allowing water to displace the cleaved DNA and proteins. Similarly, when surface-accessible glycoprotein carbohydrate chains are cleaved, by beta-galactosidase, for example, the underlying sialic acid/sulfate-calcium association becomes singular, i.e., is no longer connected to the protein backbone, and no longer a component of a cooperative structure and is effectively displaced by the abundant water. However, surprisingly this function can also be served by the presence of proteases that perform proteolysis and facilitate breakdown of glycoproteins, thus forgoing the need for beta-galactosidase. As the surface-accessible nucleic acids and glycoproteins are cleaved and displaced, the nucleic acids and glycoproteins that were beneath the now displaced cleaved nucleic acids and glycoproteins are accessible to the DNase or RNase and protein-cleaving enzymes that repeat with progressive cycles of cleavage and displacement, thereby disintegrating the three-dimensional plaque and calculus structure, and thus disintegrating the plaque and calculus.

Thus, in one aspect a dental composition according to the present disclosure comprises at least two calculus targeting enzymes: a first selected from an enzyme or other biocatalyst having a catalytic activity that hydrolyzes the phosphate ester bonds in nucleic acids including DNA and RNA; and a second enzyme or other biocatalyst having a catalytic activity that hydrolyzes proteins and any combination thereof. In various aspects, the enzyme that hydrolyzes proteins is a chymotrypsin or a functional variant or derivative thereof.

In a composition as disclosed, at least one calculus targeting enzyme or other biocatalyst can be a DNase and/or an RNase having a catalytic activity that hydrolyzes the phosphate ester bonds in DNA and RNA. In one aspect, the at least one calculus targeting enzyme is a DNase, for example DNase I. In another aspect, the at least one calculus targeting enzyme is an RNase. In another aspect, the at least one calculus targeting enzyme is a nuclease which is both a DNAse and an RNase, for example NuCLEANase Food Grade (commercially available from c-LEcta GmbH of Leipzig, Germany).

The amount of the nuclease (DNase and/or RNase) can be varied in amount from about 50,000 Kunitz units/mL to about 800,000 Kunitz units/mL. For example, in various aspects, a nuclease is present in a composition an amount of about 100,000 Kunitz units/mL, about 200,000 Kunitz units/mL, about 300,000 Kunitz units/mL, about 400,000 Kunitz units/mL, about 500,000 Kunitz units/mL, about 600,000 Kunitz units/mL, about 700,000 Kunitz units/mL, or about 750,000 Kunitz units/mL. In some aspects, the amount of nuclease can be about 200 Kunitz units/mL to about 100,000 Kunitz units/mL. For example, in various aspects, a nuclease is present in a composition an amount of about 200 Kunitz units/mL, about 300 Kunitz units/mL, about 400 Kunitz units/mL, about 500 Kunitz units/mL, about 600 Kunitz units/mL, about 700 Kunitz units/mL, about 800 Kunitz units/mL, about 900 Kunitz units/mL, about 1000 Kunitz units/mL, about 1500 Kunitz units/mL, about 2,000 Kunitz units/mL, about 2500 Kunitz units/mL, about 3000 Kunitz units/mL, about 4000 Kunitz units/mL, about 5000 Kunitz units/mL, about 6000 Kunitz units/mL, about 7000 Kunitz units/mL, about 8000 Kunitz units/mL, about 9000 Kunitz units/mL, or about 100,000 Kunitz units/mL.

In some aspects, a nuclease from any suitable source organism can be used. In some aspect, the nuclease can be from a recombinant source. In some aspects the nuclease can be synthetic. A non-limiting example of a DNase is Bovine pancreatic deoxyribonuclease (i.e., “Deoxyribonuclease I”; SEQ. ID. NO.: 1) or a functional variant or derivative thereof. In some aspects a DNase comprises a polypeptide sequence at least about 60% to about 75%, or about 75% to about 80%, or about 80% to about 85%, or about 85% to about 90%, or about 90% to about 95%, or about 95% to about 100% identical to SEQ ID NO: 1. In some aspects, the polypeptide is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 1.

In another aspect, the composition further comprises a protease, for example chymotrypsin. Chymotrypsin also referred to as EC 3.4.21.1, chymotrypsins A and B, alpha-chymar ophth, avazyme, chymar, chymotest, enzeon, quimar, quimotrase, alpha-chymar, alpha-chymotrypsin A, alpha-chymotrypsin is a serine protease produced by the pancreas that hydrolyzes the peptide bonds of tryptophan, leucine, tyrosine, and phenylalanine. Chymotrypsin preferentially cleaves peptide amide bonds where the side chain of the amino acid N-terminal to the scissile amide bond (the P₁ position) is a large hydrophobic amino acid (tyrosine, tryptophan, and phenylalanine). These amino acids contain an aromatic ring in their side chain that fits into a hydrophobic pocket (the S₁ position) of the enzyme. Chymotrypsin also hydrolyzes other amide bonds in peptides at slower rates, particularly those containing leucine and methionine at the P₁ position.

Chymotrypsin, in the body is produced as an inactive precursor chymotrypsinogen (for example, bovine chymotrypsinogen, SEQ. ID. NO: 2). It is activated into its active form by another enzyme called trypsin. This active form is called π-chymotrypsin and is used to create α-chymotrypsin. Trypsin cleaves the peptide bond in chymotrypsinogen between arginine-15 and isoleucine-16. This creates two peptides within the π-chymotrypsin molecule, held together by a disulfide bond. One of the π-chymotrypsins acts on another by breaking a leucine and serine peptide bond. The activated π-chymotrypsin reacts with other π-chymotrypsin molecules to cleave out two dipeptides, which are, serine-14-arginine-15 and threonine-147-asparagine-148. This reaction yields the α-chymotrypsin comprising sequences chymotrypsin A, chymotrypsin B and chymotrypsin C (SEQ ID NOs: 3, 4, 5 respectively), that are linked via disulfide bonds to form active α-chymotrypsin.

In some aspects, chymotrypsin from any suitable source organism can be used. In some aspect, the chymotrypsin can be from a recombinant source. In some aspects the chymotrypsin can be synthetic. In some aspects, the composition comprises a polypeptide comprising one or more of SEQ ID NO: 3, 4 or 5 or variants or derivatives thereof. In some aspect, the chymotrypsin is a recombinant chymotrypsin, which has been optimized for commercial production or commercial use. In some aspects, the recombinant chymotrypsin is optimized for use in dental products. In some aspects the composition comprises a polypeptide sequence at least about 60% to about 75%, or about 75% to about 80%, or about 80% to about 85%, or about 85% to about 90%, or about 90% to about 95%, or about 95% to about 100% identical to one or more of SEQ ID NOs: 3-5. In some aspects, the composition comprises a polypeptide that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 3-5.

In some aspects, the amount of chymotrypsin is present in functional units based on the rate of hydrolysis of a chromogenic substrate in an amount of about 100 to about 5,000, or about 500 to about 4,000, or about 1000 to about 3000, or 1,500, or 1,600, or 1,700, or 1,800, or 1,900, or 2,000, or 2,100, or 2,200, or 2,300, or 2,400, or 2,500, or 2,600, or 2,700, or 2,800, or 2,900 or 3,000 units/mL.

In some aspects, the dental composition may comprise additional proteolytic enzymes. Non-limiting example of proteolytic enzymes include Aminopeptidase M, Bromelain, Carboxypeptidase A, Carboxypeptidase B, Carboxypeptidase Y, Cathepsin C, Chymotrypsin, Collagenase, Dispase, Endoproteinase Arg-C, Endoproteinase Asp-N, Endoproteinase Glu-C, Endoproteinase Lys-C, Enterokinase, Factor Xa, Ficin, Kallikrein, Papain, Pepsin, Plasmin, Pronase, Proteinase K, Subtilisin, Thermolysin, Thrombin or Trypsin.

In some aspects, the dental composition can further comprise a calculus targeting enzyme that hydrolyses carbohydrate polymers of glycoproteins. In various aspects, the calculus targeting enzyme that hydrolyses carbohydrate polymers of glycoproteins is not beta-galactosidase.

In some aspects, the dental composition disclosed herein comprises an effective amount of a nuclease (DNase and/or RNase) and an effective amount of a chymotrypsin, or a functional variant or derivative thereof. In some aspects, the dental composition disclosed herein comprises an effective amount of a DNase and an effective amount of a chymotrypsin, or a functional variant or derivative thereof. In some aspects, the dental composition disclosed herein comprises an effective amount of a DNase I and an effective amount of a chymotrypsin, or a functional variant or derivative thereof. In some aspects, the dental composition disclosed herein comprises an effective amount of a nuclease (DNase and/or RNase), an effective amount of a chymotrypsin, or a functional variant or derivative thereof, an orally acceptable additive, carrier or excipient or any combination thereof. In some aspects, the dental composition disclosed herein comprises an effective amount of a DNase, an effective amount of a chymotrypsin, or a functional variant or derivative thereof, an orally acceptable additive, carrier or excipient or any combination thereof.

In some aspects, the dental composition disclosed herein comprises an effective amount of a DNase I, an effective amount of a chymotrypsin, or a functional variant or derivative thereof, an orally acceptable additive, carrier or excipient or any combination thereof.

In some aspects, the dental composition disclosed herein does not comprise β-galactosidase. In some aspects, the dental composition essentially consists of an effective amount of a nuclease (DNase and/or RNase) and an effective amount of a chymotrypsin, or a functional variant or derivative thereof. In some aspects, the dental composition essentially consists of an effective amount of a DNase and an effective amount of a chymotrypsin, or a functional variant or derivative thereof. In some aspects, the dental composition essentially consists of an effective amount of DNase I and an effective amount of a chymotrypsin, or a functional variant or derivative thereof.

Units of an enzyme as disclosed herein should be understood according to customary usage in the field for each enzyme. For example, for DNase, 1 unit is defined as the amount of enzyme required to produce an increase in absorbance at 260 nm of 0.001/min/mL at 25° C. of highly polymerized DNA, under conditions of HCl, pH 7.5, 50 mM MgCl₂, and 13 mM CaCl₂). For chymotrypsin, 1 unit is the amount of enzyme required to hydrolyze 1 micromole of N-benzoyl-L-tyrosine ethyl ester per minute at pH 7.8 at 25° C. For Proteinase K, 1 unit is the amount of enzyme required to digest urea-denatured hemoglobin at pH 7.5, 37° C., per minute, to produce absorbance equal to that of 1.0 μmol of L-tyrosine using Folin & Ciocalteu's phenol reagent (6). (See, e.g., www.worthington-biochem.com/PROK/cat.html). Those of skill in the art will appreciate how to define a unit for other proteolytic enzymes. In the examples below, the enzymes amylase and DNase-free RNase are used as controls.

Stable enzymes for preparing the dental compositions and formulations can be readily obtained as a purified, lyophilized powder from a commercial enzyme supplier such as Worthington Biochemical Corp. (Lakewood, NJ). Enzymes may be sourced from animal tissue such as animal (e.g., bovine) pancreas, or produced using recombinant methods. The lyophilized powder is dissolved in an aqueous solvent, which may be water. Sufficient solvent is added to the commercially supplied vial of lyophilized powder, to fill the vial about half full, and the remainder of the vial filled with glycerol to produce a reasonably shelf stable 50/50, water/glycerol solution. The solution can be maintained as such in a refrigerator for at least a few weeks. A plastic or glass pipette or other instrument is used to extract about 50 μl to about 200 μl of each enzyme solution. The enzyme or other biocatalyst solution, or combination of enzyme or other biocatalyst solutions, or a composition or formulation comprising the enzymes is then applied to the teeth using any of a variety of known oral application methods or tools, with particular attention paid to the gingival border. For example, the enzyme solution(s) or a composition or formulation containing one or more enzymes can be applied to a toothbrush, or preferably to a smaller interdental pick with a brush. Alternatively, multiple enzymes can be prepared as described, and then combined in a single solution and then applied to the applicator brush, or each enzyme solution can be applied separately to the brush. Alternatively, a composition or formulation comprising one or more enzymes can be prepared as described, and then applied to the applicator brush. The brush is used to apply the enzyme solution to the tooth surfaces, with particular attention paid to the gingival border where calculus tends to form.

Any one or more calculus targeting enzymes can be prepared as a simple solution as detailed above, or combined with orally acceptable additives, carriers or excipients to prepare a liquid, paste or gel form that helps maintain contact of the enzyme(s) with the tooth surface for a more extended period than a liquid allows. Enzyme solutions as disclosed herein can be combined with or added to a mouthwash or toothpaste composition as known in the art. Non-limiting examples of orally acceptable additives, carriers or excipients are generally as known in the art and include thickeners or gelling agents, binders, stabilizers, preservatives, flavorings, fluoride salts, surfactants, abrasives, tartar control agents, calcium sequestrants, and colorings. Additives such as flavorings and colorings can be generally as known in the art and readily commercially available.

Non-limiting examples of binders include carboxyvinyl polymers (such as polyacrylic acids cross-linked with polyallyl sucrose or polyallyl pentaerythritol), hydroxyethyl cellulose, hydroxypropyl cellulose, water soluble salts of cellulose ethers (such as sodium carboxymethyl cellulose and sodium carboxymethyl hydroxyethyl cellulose), natural gums (such as carrageenan, gum karaya, guar gum, xanthan gum, gum arabic, and gum tragacanth).

Non-limiting examples of thickeners and gelling agents are gellan gum (low acyl or high acyl), glycerol, silica, guar gum, xanthan gum, polyethylene glycols, polyvinyl pyrrolidones and co-polymers thereof, polylactic acids, polyglyocolic acids, long chain fatty acid alcohols, cellulose-based polymers and acrylate polymers.

Non-limiting examples of carriers are orally acceptable alcohols such as ethanol, isopropanol and glycerol. Non-limiting examples of tartar control agents are generally as known used in readily commercially available dentifrice products, such as pyrophosphates and their salts, polyphosphates, polyphosphonates and mixtures thereof. Pyrophosphate salts include dialkali and tetra-alkali metal pyrophosphate salts and mixtures thereof. Non-limiting examples of antiseptics and preservatives are quaternary ammonium salts, polymers thereof, chlorhexidine and salts thereof, polyhexamethylene biguanide, octenidine, organic acids, chelating agents for example a calcium chelating agent (e.g., Ethylenediaminetetraacetic acid (EDTA)), essential oils, and parabens. Non-limiting examples of antibiotics are penicillin and tetracyclin. Non-limiting examples of orally acceptable abrasives are silica, including fumed silica, or other inorganic particles, synthetic polymer particles, or organic particles such as plant-derived particles.

Any orally acceptable surfactant, most of which are anionic, nonionic or amphoteric can be used. In some aspects, the surfactant choice and amount are such that it does not interfere with enzyme activity. Non-limiting examples of nonionic surfactants include ceterareth, steareth, polyethoxylated fatty acid sorbitan esters, ethoxylated fatty acids, esters of polyethylene glycol, ethoxylates of fatty acid monoglycerides and diglycerides, and ethylene oxide/propylene oxide block polymers. In some aspects, the surfactant is selected from polyethylene glycol ethers of fatty alcohols in particular polyethylene glycol ethers having from 20 to 40 ethylene oxide groups per unit. In some exemplary aspects the surfactant is Ceteareth-25 or Steareth-30. One or more surfactants are optionally present in a total amount of about 0.000001% to about 5% depending on the type of formulation and surfactant used. In some aspects, the surfactant amount is about 0.000001% to about 0.00001%, or about 0.00001% to about 0.0001%, or about 0.0001% to about 0.001%, or about 0.001% to about 0.01%, or about 0.01% to about 1%, or about 1% to about 5% by weight of the composition.

In some aspects, the dental composition or formulation further comprises water and/or polyhydric alcohol. Typical polyhydric alcohols for use in the oral care composition, particularly a dentifrice or toothpaste composition include humectants such as glycerol, sorbitol, polyethylene glycol, polypropylene glycol, propylene glycol, xylitol (and other edible polyhydric alcohols), hydrogenated partially hydrolyzed polysaccharides and mixtures thereof. In some aspects the polyhydric alcohol is glycerol and/or sorbitol. In some aspects, the amount of water and/or polyhydric alcohol depends on the nature of the formulation. In some exemplary aspects, the amount of water and/or polyhydric alcohol will be at least 10 wt %, or at least 20 wt %, or at least 30 wt %, or at least 40 wt % percent, or at least 50 wt %, or at least 60 wt %, or at least 70 wt %, or at least 80 wt % based on the total weight of the composition. In some aspects, the level of water and/or polyhydric alcohol will be less than 95 wt % of the total composition, or less than 90 wt %, or less than 85 wt % or less than 80 wt %.

In some aspects, the formulation or composition of the present disclosure comprises about 5% to about 90% glycerol. In some aspects, the glycerol can be present in the composition or formulation at about 5%, about 10%, about 15%, about 20%, about 25%, about 30%, about 35%, about 40%, about 45%, about 50%, about 55%, about 60%, about 65%, about 70%, about 75%, about 80%, about 85%, or about 90%. In some aspects, the glycerol can be present in the composition or formulation at about 10% to about 20%.

In some aspects, the formulation or composition of the present disclosure comprises about 0.0000001% to about 0.01% Ceteareth-25. In some aspects, the Ceteareth-25 can be present in the composition or formulation at about 0.0000001%, about 0.0000005%, about 0.000001%, about 0.000005%, about 0.00001%, about 0.00005%, about 0.0001%, about 0.0005%, about 0.001%, or about 0.01%. In some aspects, about 0.000001% to about 0.00001% of Ceteareth-25 can be present in the composition or formulation.

In some aspects, the formulation or composition of the present disclosure comprises about 0.01% to about 10% Xanthan gum. In some aspects, Xanthan gum can be present in the formulation or composition at about 0.001%, about 0.005%, about 0.01%, about 0.05%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10%. In some aspects, about 0.1% to about 0.5% Xanthan gum can be present in composition or formulation.

In some aspects, the formulation or composition of the present disclosure comprises about 0.1% to about 30% sorbitol. In some aspects, sorbitol can be present in the composition or formulation at about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%. In some aspects, about 1% to about 10% sorbitol can be present in the composition or formulation.

In some aspects, the formulation or composition of the present disclosure comprises about 0.1% to about 50% silica. In some aspects, the silica is present in the composition or formulation at about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%.

In some aspects, the formulation or composition of the present disclosure can further comprise a solvent, for e.g., water. In some aspects, water can be present in the composition or formulation at about 0.01% to about 99%. In some aspects, water can be present at about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%.

In some aspects, the pH of the compositions disclosed herein can be adjusted with pharmaceutically acceptable acids, bases, or buffers. The pH of the compositions disclosed herein can be optimally in the range of about 6.0 to about 9.5, preferably in the range of about 6.1 to about 7.0 (e.g., about 6.1 to about 6.3 or about 6.7 to about 7.0).

In some aspects, the compositions or formulations provided herein can be prepared and used in various forms applicable to the mouth such as in the form of a dentifrice including toothpaste, toothpowder, tooth gel, mouthwash, mouth spray, troches, chewing gums, dental pastes, gingival massage creams, gargle tablet, or liquid formulations. In some aspects, the formulation is a dentifrice. The term “dentifrice” generally denotes formulations which are used to clean the surfaces of the oral cavity. The dentifrice is an oral composition that is not intentionally swallowed for purposes of systemic administration of therapeutic agents, but is applied to the oral cavity, used to treat the oral cavity and then expectorated. In some aspects, the dentifrice is used in conjunction with a cleaning implement such as a toothbrush, usually by applying it to the bristles of the toothbrush and then brushing the accessible surfaces of the oral cavity. In some aspects, the dentifrice is in the form of a paste or a gel (or a combination thereof).

A dentifrice composition according to the disclosure can contain, as the aqueous continuous phase, a mixture of water and polyhydric alcohol in various relative amounts, with the amount of water generally ranging from 10 to 50% by weight (based on the total weight of the dentifrice) and the amount of polyhydric alcohol generally ranging from about 20% to about 60% by weight (based on the total weight of the dentifrice).

The oral care composition, in particular toothpaste, or dentifrice compositions may further comprise additional abrasive materials. In some aspects, abrasive materials may be present in an amount of from about 0.5% to about 75%, about 3% to about 60% by weight based on the total weight of the paste. Suitable abrasive cleaning agents include silica xerogels, hydrogels and aerogels and precipitated particulate silicas; calcium carbonate, dicalcium phosphate, tricalcium phosphate, calcined alumina, sodium and potassium metaphosphate, sodium and potassium pyrophosphates, sodium tri-metaphosphate, sodium hexametaphosphate, particulate hydroxyapatite and mixtures thereof. In some aspects the abrasive material is silica, present in an amount of at least about 10% to 15%, or 15% to 20% or 20% to 25%, or 25% to 30% or 30% to 35%, or 35% to 40%, or 40% to 45%, or 45% to 50%, or 50% to 55%, or 55% to 60%, or 60% to 70% or 70% to 80% by weight.

In some aspects, the compositions of the present disclosure may comprise a zinc ion. Preferably the sources of zinc ions are zinc chloride, zinc acetate, zinc gluconate, zinc sulphate, zinc fluoride, zinc citrate, zinc lactate, zinc oxide, zinc monoglycerolate, zinc tartrate, zinc pyrophosphate zinc maleate and mixtures thereof.

In some aspects, the compositions of the present disclosure may also contain further optional ingredients customary in the art such as fluoride ion sources, buffers, flavoring agents, sweetening agents, coloring agents, opacifying agents, preservatives, ant sensitivity agents, bleaching and antimicrobial agents.

In some aspects, composition of the present disclosure may be substantially liquid in character, such as a mouthwash or rinse. In such a preparation the vehicle is typically a water-alcohol mixture desirably including a humectant as described below. Generally, the weight ratio of water to alcohol is in the range of from about 1:1 to about 20:1. The total amount of water-alcohol mixture in this type of preparation is typically in the range of from about 70% to about 99.9% by weight of the preparation. The alcohol is typically ethanol or isopropanol.

In some aspects, the composition may be a solid, semi-solid, gel, or paste in character, such as toothpowder, a dental tablet or a toothpaste (dental cream) or gel dentifrice. The vehicle of such solid or pasty oral preparations generally contains dentally acceptable polishing material. Non-limiting examples of polishing materials can include water-insoluble sodium metaphosphate, potassium metaphosphate, tricalcium phosphate, dihydrated calcium phosphate, anhydrous dicalcium phosphate, calcium pyrophosphate, magnesium orthophosphate, trimagnesium phosphate, calcium carbonate, hydrated alumina, calcined alumina, aluminium silicate, zirconium silicate, silica, bentonite, and mixtures thereof. Other suitable polishing material can include the particulate thermosetting resins such as melamine-, phenolic, and urea-formaldehydes, and cross-linked polyepoxides and polyesters. In some aspects, polishing materials can include crystalline silica having particle sizes of up to about 5 microns, a mean particle size of up to about 1.1 microns, and a surface area of up to about 50,000 cm²/g, silica gel or colloidal silica, and complex amorphous alkali metal aluminosilicate.

The polishing material is generally present in the solid or pasty compositions in weight concentrations of about 10% to about 99%. In some aspects, the polishing material can be present in amounts from about 10% to about 75% in toothpaste, and from about 70% to about 99% in toothpowder. In toothpastes, when the polishing material is silicious in nature, it is generally present in an amount of about 10-30% by weight. Other polishing materials are typically present in amount of about 30-75% by weight.

In a toothpaste, the liquid vehicle may comprise water and humectant typically in an amount ranging from about 10% to about 80% by weight of the preparation. Glycerine, propylene glycol, sorbitol and polypropylene glycol exemplify suitable humectants/carriers. In some aspects, liquid mixtures of water, glycerin and sorbitol can be used. In clear gels where the refractive index is an important consideration, about 2.5%-30% w/w of water, 0% to about 70% w/w of glycerin and about 20% to about 80% w/w of sorbitol can be employed.

Toothpaste, creams and gels typically contain a natural or synthetic thickener or gelling agent in proportions of about 0.1 to about 10, preferably about 0.5 to about 5% w/w. A suitable thickener can be a synthetic hectorite, a synthetic colloidal magnesium alkali metal silicate complex clay available for example as Laponite (for e.g. CP, SP 2002, D) marketed by Laporte Industries Limited. Laponite D is, approximately by weight 58.00% SiO₂, 25.40% MgO, 3.05% Na₂O, 0.98% Li₂O, and some water and trace metals. Other suitable thickeners, non-limiting examples can include Irish moss, iota carrageenan, gum tragacanth, starch, polyvinylpyrrolidone, hydroxyethylpropylcellulose, hydroxybutyl methyl cellulose, hydroxypropyl methyl cellulose, hydroxyethyl cellulose (e.g., available as Natrosol), sodium carboxymethyl cellulose, and colloidal silica such as finely ground Syloid (e.g. 244). Solubilizing agents may also be included such as humectant polyols such propylene glycol, dipropylene glycol and hexylene glycol, cellosolves such as methyl cellosolve and ethyl cellosolve, vegetable oils and waxes containing at least about 12 carbons in a straight chain such as olive oil, castor oil and petrolatum and esters such as amyl acetate, ethyl acetate and benzyl benzoate.

In addition, the toothpaste composition of the present disclosure can further comprise medicinal ingredients. Common toothpaste compositions comprise appropriate medicinal ingredients according to use purpose, and recently fluorides that form fluorine films on tooth to make tooth resistant to acids such as lactic acids, metabolites of dental caries-causing bacteria, are recognized to be indispensable for a toothpaste composition. In some aspects, the composition can comprise sodium fluoride or sodium monofluoro phosphate alone or in combination. In addition, in order to prevent periodontitis, water-soluble salts such as sodium chloride, sodium bicarbonate having high osmotic pressure, aminocaproic acid, alantoin and alantoin derivatives and various vitamins can be simultaneously used.

In addition, the toothpaste composition of the present disclosure can further comprise a binding agent. A binding agent, which prevents separation of solid powder ingredients and liquid ingredients, is indispensable for an ointment type toothpaste, and any water-soluble macromolecules can be used. Sodium carboxymethyl cellulose synthesized from cellulose of trees, carrageenan extracted from seaweeds and xanthan gum obtained from metabolism of microorganisms, etc. are generally used.

In addition, the toothpaste composition of the present disclosure can further comprise a foaming agent. A foaming agent increases use feels of product, aids cleaning and facilitates dispersion and penetration of medicinal ingredients and decreases surface tension thereby easily separating extraneous matter. As a foaming agent, anionic surfactant sodium lauryl sulfate is predominantly used, and nonionic surfactant polyoxyethylene polyoxypropylene copolymer (poloxamer), polyoxyethylene hardened castor oil, polyoxyethylene sorbitan fatty acid ester, etc. can be supplementarily used according to the properties of preparations.

Various other materials can be incorporated in the oral preparations of this disclosure such as whitening agents, preservatives, silicones, chlorophyll compounds and/or ammoniated material such as urea, diammonium phosphate, and mixtures thereof. These adjuvants, where present, can be incorporated in the preparations in amounts which do not substantially adversely affect the properties and characteristics desired.

Any suitable flavoring or sweetening material can also be employed. Non-limiting examples of suitable flavoring constituents include flavoring oils, for e.g. oil of spearmint, peppermint, wintergreen, sassafras, clove, sage, eucalyptus, marjoram, cinnamon, lemon, and orange, and methyl salicylate. Suitable sweetening agents can include sucrose, lactose, maltose, sorbitol, xylitol, sodium cyclamate, perillartine, AMP (aspartyl phenyl alanine, methyl ester), saccharine, and the like. Suitably, flavour and sweetening agents can each or together comprise from about 0.1% to 5% more of the preparation.

The compositions of this disclosure can also be incorporated in lozenges, or in chewing gum or other products, for e.g. by stirring into a warm gum base or coating the outer surface of a gum base, illustrative examples of which include jelutong, rubber latex, vinylite resins, etc., desirably with conventional plasticizers or softeners, sugar or other sweeteners or such as glucose, sorbitol and the like.

In some aspects, the oral preparations can be sold or otherwise distributed in suitable labelled packages. For e.g., a bottle of mouth rinse will have a label describing it, in substance, as a mouth rinse or mouthwash and having directions for its use; and a toothpaste, cream or gel will usually be in a collapsible tube, typically aluminum, lined lead or plastic, or other squeeze, pump or pressurized dispenser for metering out the contents, having a label describing it, in substance, as a toothpaste, gel or dental cream.

In a further aspect, the disclosure provides compositions including compositions described above and a source of fluoride ions together with a carrier. Such compositions may be a dental, compositions in the form of a gel, liquid, solid, powder, cream or lozenge.

In some aspects, the disclosure encompasses an oral hygiene device comprising the disclosed composition or formulation. In some aspects the dental composition or formulation can be used with a dental tray, a capsule applicator, an oral pick or a dental floss.

In some aspects, the dental tray can include an article shaped to at least partially overlay one or more teeth, gums, or dental implants. In some aspects, a dental tray can have an arch shape, a circular, or a semi-circular shape. In some aspects, a dental tray may be a dental aligner (for e.g. orthodontic aligner or retainer), a night guard, a mouth guard, a treatment tray, complete or partial dentures, a tooth cap, or the like. In some aspects, dental tray may include an elastic polymeric material that generally conforms to a patient's teeth, and may be transparent, translucent, or opaque.

In some aspects, the dental tray of the present disclosure can be prepared using a casting and curing method. In some aspects, the dental tray can be prepared by a method including the steps of (a) preparing a polymerizable composition; (b) depositing the polymerizable composition onto a master negative molding surface in an amount sufficient to fill the cavities of the master; (c) filling the cavities by moving a bead of the polymerizable composition between a preformed base (such as a PET film) and the master, at least one of which is flexible; and (d) curing the composition.

In some aspects, the dental tray can be prepared using a polymeric material that may include, for example, one or more of amorphous thermoplastic polymers, semi-crystalline thermoplastic polymers and transparent thermoplastic polymers such as polycarbonate, thermoplastic polyurethane, acrylic, polysulfone, polypropylene, polypropylene/ethylene copolymer, cyclic olefin polymer/copolymer, poly-4-methyl-1-pentene or polyester/polycarbonate copolymer, styrenic polymeric materials, polyamide, polymethylpentene, polyetheretherketone, polyurethanes, polyolefins including metallocene polyolefins, polyesters such as elastomeric polyesters (e.g., Hytrel), biodegradable polyesters such as polylactic, polylactic/glycolic acids, copolymers of succinic acid and diols, and the like, fluoropolymers including fluoroelastomers, polyacrylates, polymethacrylates, or any combinations thereof. In another aspect, the material can be chosen from clear or substantially transparent semi-crystalline thermoplastic, crystalline thermoplastics and composites, such as polyamide, polyethylene terephthalate, polybutylene terephthalate, polyester/polycarbonate copolymer, polyolefin, cyclic olefin polymer, styrenic copolymer, polyetherimide, polyetheretherketone, polyethersulfone, polytrimethylene terephthalate, and mixtures and combinations thereof. In some aspects, material can be a polymeric material chosen from polyethylene terephthalate, polyethylene terephthalate glycol, polycyclohexylenedimethylene terephthalate glycol, and mixtures and combinations thereof. In some aspects, dental tray can comprise a material including styrene-acrylonitrile, cellulose acetate butyrate, cellulose acetate propionate, cellulose triacetate, polyether sulfone, polymethyl methacrylate, polyurethane, polyester, polycarbonate, polyvinyl chloride, polystyrene, polyethylene naphthalate, copolymers or blends based on naphthalene dicarboxylic acids, polycyclo-olefins, polyimides, and glass. In some aspects, the dental tray may be comprised of a biodegradable material. Non-limiting examples of suitable biodegradable polymers include polycarboxylic acid; polyanhydrides such as maleic anhydride polymers; polyorthoesters; poly-amino acids; polyethylene oxide; polyphosphazenes; polylactic acid, polyglycolic acid, and copolymers and mixtures thereof such as poly(L-lactic acid) (PLLA), poly(D,L-lactide), poly(lactic acid-co-glycolic acid), and 50/50 weight ratio (D,L-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and mixtures thereof; polycarbonates such as tyrosine-derived polycarbonates and acrylates, polyiminocarbonates, and polydimethyltrimethylcarbonates; cyanoacrylate; calcium phosphates; polyglycosaminoglycans; macromolecules such as polysaccharides (including hyaluronic acid, cellulose, and hydroxypropylmethyl cellulose; gelatin; starches; dextrans; and alginates and derivatives thereof, proteins and polypeptides; and mixtures and copolymers of any of the foregoing. The biodegradable polymer can also be a surface erodible polymer such as polyhydroxybutyrate and its copolymers, polycaprolactone, polyanhydrides (both crystalline and amorphous), and maleic anhydride. In some aspects, the dental tray can be made of a single polymeric material or can include multiple layers of different polymeric materials.

In certain aspects, compositions or formulations provided herein can be prepared and used in various forms applicable to an animal. In such aspects, the compositions or formulations provided herein can be prepared and used in various forms, non-limiting examples of which can include pet toothbrush and pet toothpaste, chew sticks, rubber toys, chew toys, treats, bite or nibbling toys, pet food, pet treats, pet dental gum, nugget, powder, or dental device (for e.g., dental tray). Compositions or formulations provided herein can be incorporated to any of the disclosed forms applicable to an animal using known methods in the art.

### (II) Methods of Removing Dental Calculus

The present disclosure encompasses methods for reducing or removing dental calculus from a tooth surface in a subject in need thereof. In some aspects, the subject is a human or a non-human animal, including but not limited to a feline, canine, bovine, equine or primate subject.

In some aspects, the method for reducing or removing calculus comprises for example contacting a tooth surface of a subject, with an effective amount of at least two calculus targeting enzyme as disclosed herein. The contacting may be for example by applying a solution (e.g., an aqueous solution), a dentifrice or a dental composition or dental formulation as disclosed herein to the tooth surface, waiting for a period of time and then rinsing the mouth out, typically with water. It will be appreciated that the amount of time can vary depending on a range of factors including the degree and severity of the calculus build-up, the individual being treated, whether the treatment is taking place in a professional office by a dental professional or at home, and other factors. For example, the solution can be applied to the tooth surface for a period of less than about 60 minutes, less than about 30 minutes, less than about 15 minutes, less than about 15 minutes, less than less than about 10 minutes, less than about 5 minutes, less than about 2 minutes, less than about 1 minute, or about 30 seconds.

The contacting may be performed on a repeated and/or regular basis, such as once or twice or more often on a daily basis, once every two days once every three days, once every four days, once every five days, once every six days, once weekly, once biweekly, once every three weeks, or once monthly, or once or twice annually at approximately regular spaced intervals. Further, the contacting may be before, during/in combination with, or after, contacting or treatment with an oral care agent such as a toothpaste or mouth rinse. Following the contacting with an enzyme or other biocatalyst solution or composition, disintegrated or loosened calculus can be further removed from the tooth surface and oral cavity by rinsing with water or another agent, manual scaling or scraping, brushing, and/or swabbing. In some aspects, the method can comprise use of the dental composition provided herein along with additional dental treatment, routines and/or procedures.

In some aspects, the disclosure encompasses a method for reducing or removing calculus dentalis in a subject in need thereof, the method comprising contacting a tooth surface with an effective amount of a composition. In such aspects, the method for reducing or removing calculus dentalis in a subject in need thereof, comprises contacting a tooth surface with an effective amount of a composition comprising DNAse I or a functional variant or derivatives thereof, and chymotrypsin or a functional variant or derivative thereof. In some aspects, the method reduces or removes calculus dentalis in a subject by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100% or more, compared to a subject or average of a population of subjects not using the disclosed composition or formulation. In some aspects, the disclosed formulation or composition reduces or removes calculus by at least 40%.

In some aspects, the formulation or composition of the present disclosure can be used in a method for preventing calculus in a subject in need thereof. In such aspects, the preventing calculus dentalis in a subject in need thereof, comprises contacting a tooth surface with an effective amount of a composition comprising DNAse I or a functional variant or derivatives thereof, and chymotrypsin or a functional variant or derivative thereof. In some aspects, the method prevents calculus dentalis in a subject by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100% or more, compared to a subject or average of a population of subjects not using the disclosed composition or formulation.

In some aspects, the formulation or composition of the present disclosure can be used in a method of improving the mouthfeel in a subject in need thereof. In such aspects, the method of improving the mouthfeel in a subject in need thereof, comprises contacting a tooth surface with an effective amount of a composition comprising DNAse I or a functional variant or derivatives thereof, and chymotrypsin or a functional variant or derivative thereof. In some aspects, the method improves the mouth-feel in a subject by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100% or more, compared to a subject or average of a population of subjects not using the disclosed composition or formulation.

In some aspects, the composition or formulation disclosed herein can be administered to the subject after a professional dental cleaning. In some aspects, the composition or formulation disclosed herein can be administered to the subject before a scheduled professional dental cleaning. In some aspects, the composition or formulation disclosed herein can be administered to the subject during a professional dental cleaning.

In some aspects, the method of administering comprises brushing with the formulation or composition of the present disclosure for about 10 seconds to about 120 seconds. In some aspects, brushing can comprise brushing with the formulation or composition for about 10 seconds, about 11 seconds, about 12 seconds, about 13 seconds, about 14 seconds, about 15 seconds, about 16 seconds, about 17 seconds, about 18 seconds, about 19 seconds, about 20 seconds, about 21 seconds, about 22 seconds, about 23 seconds, about 24 seconds, about 25 seconds, about 26 seconds, about 27 seconds, about 28 seconds, about 29 seconds, about 30 seconds, about 31 seconds, about 32 seconds, about 33 seconds, about 34 seconds, about 35 seconds, about 36 seconds, about 37 seconds, about 38 seconds, about 39 seconds, about 40 seconds, about 41 seconds, about 42 seconds, about 43 seconds, about 44 seconds, about 45 seconds, about 46 seconds, about 47 seconds, about 48 seconds, about 49 seconds, about 50 seconds, about 51 seconds, about 52 seconds, about 53 seconds, about 54 seconds, about 55 seconds, about 56 seconds, about 57 seconds, about 58 seconds, about 59 seconds, about 60 seconds, about 61 seconds, about 62 seconds, about 63 seconds, about 64 seconds, about 65 seconds, about 66 seconds, about 67 seconds, about 68 seconds, about 69 seconds, about 70 seconds, about 71 seconds, about 72 seconds, about 73 seconds, about 74 seconds, about 75 seconds, about 76 seconds, about 77 seconds, about 78 seconds, about 79 seconds, about 80 seconds, about 81 seconds, about 82 seconds, about 83 seconds, about 84 seconds, about 85 seconds, about 86 seconds, about 87 seconds, about 88 seconds, about 89 seconds, about 90 seconds, about 91 seconds, about 92 seconds, about 93 seconds, about 94 seconds, about 95 seconds, about 96 seconds, about 97 seconds, about 98 seconds, about 99 seconds, about 100 seconds, about 101 seconds, about 102 seconds, about 103 seconds, about 104 seconds, about 105 seconds, about 106 seconds, about 107 seconds, about 108 seconds, about 109 seconds, about 110 seconds, about 111 seconds, about 112 seconds, about 113 seconds, about 114 seconds, about 115 seconds, about 116 seconds, about 117 seconds, about 118 seconds, about 119 seconds, or about 120 seconds. In some aspects, brushing comprises brushing with the formulation or composition for about 30 seconds.

In some aspects, the method of administering comprises using a dental tray with the formulation or composition of the present disclosure for about 1 minute to about 60 minutes. In some aspects, dental tray comprises the formulation or composition can be administered for about 1 minute, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, or about 60 minutes. In some aspects, dental tray comprises the formulation or composition can be administered for about 30 minutes.

In some aspects, the composition or formulation of the present disclosure can be administered at least once a day. In some aspects, the composition or formulation can be administered at least twice a day. In some aspects, the composition or formulation can be administered at least three times a day. In some aspects, the composition or formulation can be administered more than three times a day.

In some aspects, the method comprises brushing with the formulation or composition of the present disclosure followed by brushing with a commercially available toothpaste. In some aspects, the method comprises brushing with a commercially available toothpaste, followed by brushing with the formulation or composition of the present disclosure.

In some aspects, the method comprises sequential brushing with composition or formulation of the present disclosure and use of the dental tray comprising the formulation or composition of the present disclosure. brushing with the formulation or composition of the present disclosure followed by using a dental tray with the formulation. In some aspects, the method comprises using a dental tray with the formulation or composition of the present disclosure followed by brushing with the formulation or composition of the present disclosure. In some aspects, the method comprises brushing with the formulation or composition of the present disclosure followed by using a dental tray with the formulation or the composition of the disclosure, followed by brushing with the formulation or composition of the present disclosure.

In some aspects, methods of treatment can include a single administration, multiple administrations, and repeating administration of composition or formulation disclosed herein as required for the prevention or treatment of the disease or condition from which the subject is suffering (for e.g., calculus). In some aspects, methods of treatment can include assessing a level of calculus in the subject prior to treatment, during treatment, or after treatment. In some aspects, treatment can continue until a decrease in the level of the calculus in the subject can be detected.

In some aspects, an effective dose of a composition or formulation of the present disclosure can include, but is not limited to, e.g., about 0.00001, 0.0001, 0.001, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, or 3-10,000 mg/kg/day, or according to the requirements of the particular composition or formulation.

With respect to a disclosed composition or formulation, the dosage also may be denominated in International Units (IU) per day (IU/Day) and about 100 IU/day, 200 IU/day, 300 IU/day, 400 IU/day, 500 IU/day, 600 IU/day, 700 IU/day, 800 IU/day, 900 IU/day, 1000 IU/day, 1 100 IU/day, 1200 IU/day, 1300 IU/day, 1400 IU/day, 1500 IU/day, 1600 IU/day, 1700 IU/day, 1800 IU/day, 1900 IU/day, 2000 IU/day, 2100 IU/day, 2200 IU/day, 2300 IU/day, 2400 IU/day, 2500 IU/day, 2600 IU/day, 2700 IU/day, 2800 IU/day, 2900 IU/day, 3000 IU/day, 3100 IU/day, 3200 IU/day, 3300 IU/day, 3400 IU/day, 3500 IU/day, 3600 IU/day, 3700 IU/day, 3800 IU/day, 3900 IU/day, 4000 IU/day, 4500 IU/day, 5000 IU/day, 5500 IU/day, 6000 IU/day, 6500 IU/day, 7000 IU/day, 7500 IU/day, 8000 IU/day, 9000 IU/day, 10,000 IU/day, 20,000 IU/day, 30,000 IU/day, 40,000 IU/day, 50,000 IU/day, 60,000 IU/day, 70,000 IU/day, 90,000 IU/day, 100,000 IU/day, 200,000 IU/day, 300,000 IU/day, 400,000 IU/day, 500,000 IU/day, 600,000 IU/day, 700,000 IU/day, 800,000 IU/day, 900,000 IU/day, 1,000,000 IU/day, 1,100,000 IU/day, 1,200,000 IU/day, 1,300,000 IU/day, 1,400,000 IU/day, or 1,500,000 IU/day.

Dosage amount and interval may be adjusted individually to provide a desired effect or which are sufficient to maintain therapeutic or prophylactic effect.

Specific dosage and treatment regimens for any particular subject will depend upon a variety of factors, including the activity of the specific enzymes employed, the severity of calculus and course of the calculus build up in the subject, condition or symptoms, the patient's disposition to the condition or symptoms, and the judgment of the treating physician.

An effective amount can be administered in one or more administrations, applications or dosages. A therapeutically effective amount of a formulation (i.e., an effective dosage) depends on the type of the composition or formulation selected. Moreover, treatment of a subject with an effective amount of the formulations or compositions described herein can include a single treatment or a series of treatments. For example, effective amounts can be administered at least once. The compositions can be administered one from one or more times per day to one or more times per week, including once every other day. The skilled artisan will appreciate that certain factors can influence the dosage and timing required to effectively treat a subject, including but not limited to the severity of the calculus, previous treatments, the general health or age of the subject, and other diseases present.

Following administration, the subject can be evaluated to detect, assess, or determine their level of calculus. In some instances, treatment can continue until a change (e.g., reduction) in the level of disease in the subject can be detected. Upon improvement of a patient's condition (e.g., a change (e.g., decrease) in the level of calculus in the subject), a maintenance dose of a formulation, or composition disclosed herein can be administered, if necessary. Subsequently, the dosage or frequency of administration, or both, can be reduced, e.g., as a function of the calculus build up, to a level at which the improved condition is retained. Patients may, however, require intermittent treatment on a long-term basis upon any recurrence of calculus build up.

In some aspects, the compositions disclosed herein can include other compounds, drugs, or agents used for the treatment of calculus, plaque and/or periodontal disease. For example, in some instances, compositions disclosed herein can be combined with one or more (e.g., one, two, three, four, five, or less than ten) compounds. When co-administered, formulation or compositions disclosed herein can operate in conjunction with other agents used for the treatment of calculus, plaque and/or periodontal disease to produce mechanistically additive or synergistic therapeutic effects.

### (III) Kits

A further aspect of the present disclosure provides kits comprising an amount of any of the disclosed dental compositions or formulations, or any combination thereof, as detailed above. A kit optionally includes one or more brushes, dental picks, dental trays and/or applicators for applying any of the enzyme solutions, compositions or formulations to teeth, and then removing disintegrated calculus from the tooth surfaces. Kit components can be provided in suitable containers along with other kit components such as any commercially available containers or packaging and the like. The kits provided herein generally include instructions for carrying out the methods detailed herein. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

Without intending to be bound by theory, it is believed that the present compositions and methods are so effective in breaking up and removing calculus in part because of the way in which calculus builds up and is structured. The inventor's work indicates that dental plaque has a layered structure including an internal gel or gel-like structure over which is layered one or more layers of calcium cross-bridged extracellular DNA (eDNA) and proteins in cross-linked meshwork. The internal gel or gel-like portion comprises aqueous contents similar to the contents of the salivary glands. The aqueous contents can include inorganic substances, calcium, phosphate, hydroxyapatite, proteins and glycoproteins, amylase, polyproline proteins, and mucins. Additionally, the dental plaque can comprise contents that come from non-salivary gland sources, including transglutaminase from the oral epithelial surface, and stratum corneum chymotryptic enzyme from the soft palette. Dental plaque can further include contents from non-host sources, such as bacterial cells from the oral microbiome, eDNA secreted from the microbiome, and the protein and polysaccharides from food. The outermost layers of the plaque comprise calcium cross-bridged eDNA (about 0.01%), and proteins (about 0.04%-0.05%), wherein the proteins include cross-linked meshwork of negatively charged mucin glycoproteins and transglutaminase.

Additionally, the inventor has further identified that the process of calcification of plaque can begin immediately, with the plaque becoming about 50% calcified within 2 days, to 60%-90% in about 12 days. This process of calcification is also progressive, by which successive layers are added and thereby build the calculus structure from a more 2-dimensional to a more 3-dimensional structure. While the meshwork of calcium cross-bridged eDNA and mucin glycoprotein is reversible, the meshwork formed by transglutaminase cross-linked protein is irreversible. The compositions and methods of the present disclosure have been developed to effectively target the contents that form the structure of calculi and process of its formation, to help treat or prevent calculi, as elaborated in the following examples.

### EXAMPLES

### Example 1: Anti-Calculus Enzyme Formulations with DNAse 1 and Chymotrypsin

Anti-calculus enzyme compositions containing DNAse 1 and Chymostrypsin were formulated as provided. Two batches, Formulation I and Formulation II containing different levels of nonionic surfactant Ceteareth, and water were made.

For Formulation I, the chemical ingredients disclosed in Table 1 were mixed thoroughly.

TABLE 1
Chemical ingredients for formulation I

| Stocks | Volume/amount | |
|---|---|---|
| Glycerol | 90 | ml |
| 10% Ceteareth-25 | 36 | ml |
| 2% Xanthan gum | 90 | ml |
| 80% sorbitol | 36 | ml |
| Water | 30 | ml |
| Silica | 72 | g |

About 110 ml of the combined volume was discarded. The enzymes were then mixed into the solution in batches. 5 g DNase 1 (4270 Kunitz units/mg) was added to the combined solution and mixed until uniform. 5 g chymotrypsin (66.6 units/mg) was added to the solution and mixed until uniform. This step was repeated 6 times to achieve an approximate enzyme activity of DNase of 185,652.17 units/ml and Chymotrypsin activity of about 2,895.65 units/ml. The formulation was filled into capped syringes (1 ml/syringe) and refrigerated until application.

Similarly, Formulation II was formulated by initially dissolving the chemical ingredients as provided in Table 2.

TABLE 2
Chemical ingredients for formulation II

| Stocks | Volume/amount | |
|---|---|---|
| Glycerol | 90 | ml |
| 10 Ceteareth-25 | 0.36 | ml |
| 2% Xanthan gum | 90 | ml |
| 80% sorbitol | 36 | ml |
| Silica | 72 | g |

In order to control the volume, for this formulation, the volume was made up to 200 ml with water. The enzymes were then mixed into the solution in batches. 5 g of DNAse 1 (3900 Kunitz units/mg) was added to the solution and mixed until uniform. Next, 5 g chymotrypsin (66.6 units/mg) was added to the solution and mixed until uniform. This step was repeated 6 times and an additional 30 ml of water was added. In order to obtain sufficient volume for trials, 50 ml of Formulation I was added to this formulation. The approximate DNAse 1 activity of the formulation was determined to be 183,300 Kunits/ml and chymotrypsin at 114 units of activity/ml. The formulation was filled into capped syringes (1 ml/syringe) and refrigerated until application.

Formulation II-filled syringes were used in a clinical study to evaluate calculus treatment efficacy and safety profile in human subjects.

### Example 2: In Vivo Study to Evaluate the Anti-Calculus Activity and Safety Profile of the Dental Formulations

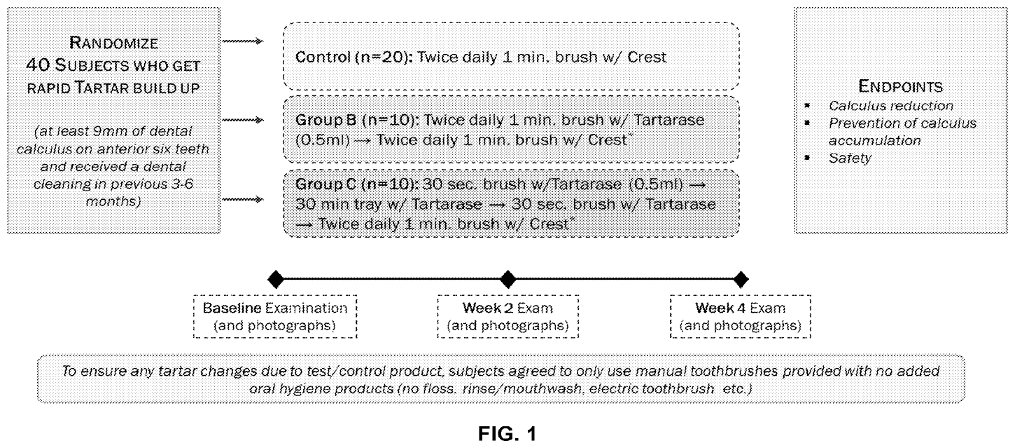

Safety and efficacy of the formulations comprising DNAse 1 and chymotrypsin to remove existing calculus deposits in subjects was studied. Inter- and intra-group comparisons over a 4-week trial period was conducted. A schematic of the study design is provided in FIG. 1.

### Recruitment

Forty subjects were recruited for these trials. Eligibility for trails was based on the following eligibility criteria.

### Inclusion Criteria

To be included in the study, each subject had to:
1. provide written informed consent to participate in the study;
2. be 18 years and older;
3. agree not to participate in any other oral/dental product studies during the course of this study and used only the assigned oral hygiene products during the entire study (including toothbrushes, toothpastes, home remedies, floss or other products like chewing gum, mouthwashes, tongue cleaners, etc.);
4. agree to refrain from the use of any elective dentistry (including a non-study dental prophylaxis) until the study had been completed;
5. agree to refrain from the use of any non-study oral hygiene and whitening products;
6. be in good general health, as determined by the investigator/designee based on a review of the health history/update for participation in the study;
7. have six mandibular anterior teeth with no crowns or veneers;
8. agree to comply with the study procedures and schedule, including the follow up visits;
9. have at least 9 mm of dental calculus on the mandibular anterior six teeth and reported that they had received a dental cleaning in previous 2-6 months.

### Exclusion Criteria

Subjects were excluded from study participation if they:
1. had a medical condition requiring antibiotic premedication prior to dental procedures;
2. regularly used chlorhexidine mouth rinse;
3. had any oral condition or pathosis that could interfere with study compliance and/or examination procedures (e.g., widespread caries, chronic neglect, advanced periodontal disease);
4. had current or history of oral cavity cancer or oropharyngeal cancer;
5. were pregnant or nursing by subject report;
6. did not brush regularly;
7. had any condition that might have made it unsafe for the subject to participate in this study, at the discretion of the investigator.

### Continuance Criteria

Subjects were withdrawn from the study and excluded from study analysis if they:
1. had participated in any other oral/dental product studies since the last visit;
2. had received any elective dentistry since the last visit;
3. had used any oral hygiene products other than the assigned study products;
4. had been unable or unwilling to comply with product usage instruction for any reason.

After subjects qualified under the inclusion/exclusion, their calculus condition was examined. Only subjects who had accumulation of at least 9 mm of calculus on the lingual surfaces of the six mandibular anterior teeth were eligible. The details of the protocol were discussed with each potential subject and written informed consent was obtained for all subjects before any activity related to the clinical investigations performed. Subjects could withdraw their consent at any time without prejudice. A subject accountability form was completed for each subject. If, for any reason, a subject did not complete the study, an explanation was entered on the Subject Accountability CRF.

### Study Visits

The following visits were performed during the study period:

### Visit 1 (Informed Consent, Baseline and Impressions):

During Visit 1 subjects received an explanation on the nature and course of the study, signed an informed consent, and received a signed copy of the consent form. After subjects signed the informed consent, the investigator performed an oral examination and reviewed the inclusion/exclusion criteria to ensure eligibility. The eligible subjects were evaluated for demographic details (including age, gender, and race), medical history and concomitant medications. Demographic information and inclusion/exclusion criteria were also obtained and captured in the appropriate Case Report Forms (CRFs). Subjects were evaluated to ensure at least 9 mm of dental calculus was present on the lingual surfaces of the six mandibular anterior teeth. Subjects who qualified were randomized to a treatment group.

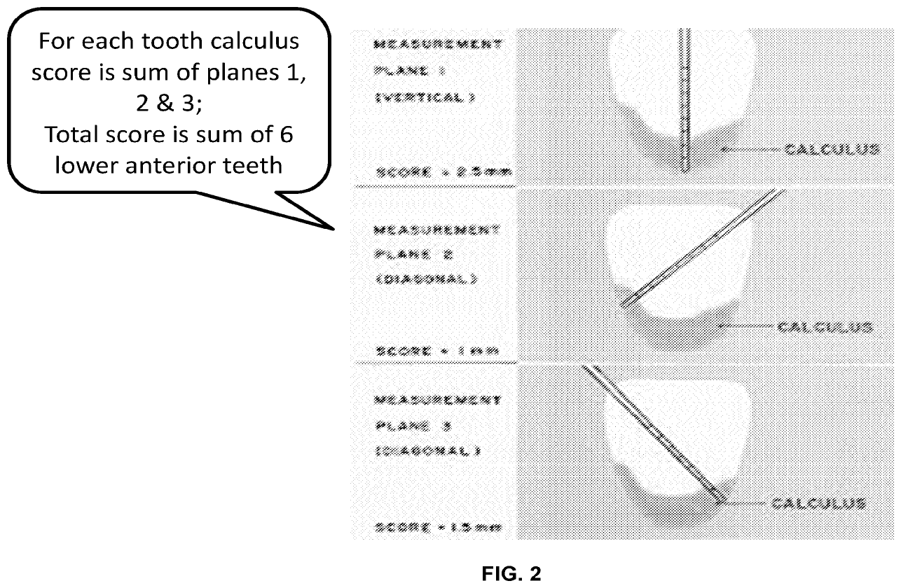

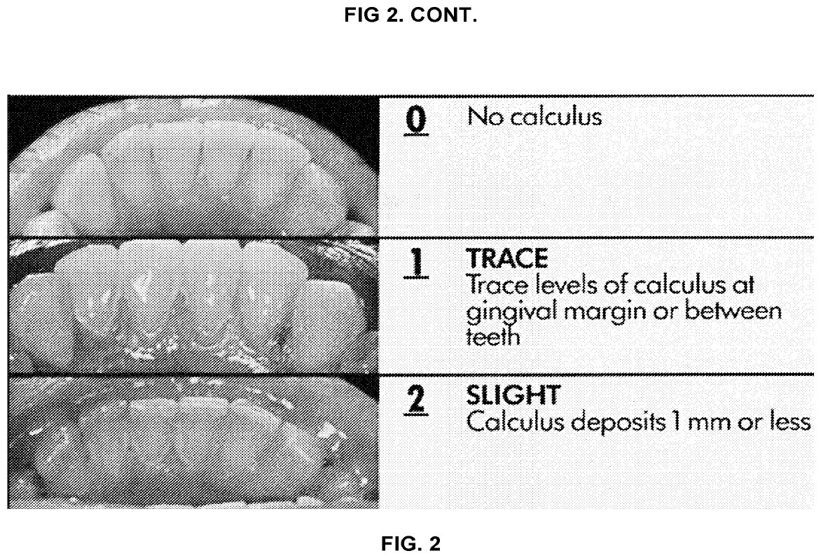

Subjects were randomly assigned to one of the three study groups by using a randomization schedule developed and maintained by an independent statistician. Subjects were stratified by baseline lingual V-MI dental calculus scores and gender (see FIG. 2).

During visit 1, all randomized subjects (with the exception of the custom tray test group who returned the following day) performed their first use, supervised at the research site.

Subjects performed at-home product use twice a day, over a period of 4 weeks. Each product use was performed according to the group instructions. The subjects received a Diary Card to document the home treatments.

Subjects who were randomized into the enzyme composition gel group with custom mouthguards received an impression at this visit. The dental impression of the mandibular teeth was used for the construction of an individual tooth shield for use during the clinical trial. The tooth shields were constructed from vacuum-formed mouthguard plastic. Each tooth shield was trimmed to include only the teeth and gingival margin and to eliminate contact with the cervical margin of each tooth to isolate the enzyme composition gel in contact with the hard tooth surface.

### Visits 2 and 3 (2-Week and 4-Week Exams):

During visits 2 and 3 (after 2 and 4 weeks of treatment, respectively), continuance criteria were assessed and documented on the appropriate CRF. Upon verification of continuing eligibility, a safety assessment was conducted via dental examination and compliance with study protocol was evaluated. An assessment of calculus deposits was made to replicate the evaluation undertaken at visit 1.

Table 3 provides an overview of the visits:

TABLE 3
Study procedure and schedule

| | Screening/Baseline | 2^{nd} and 4^{th} weeks ± 2 |
|---|---|---|
| Procedure | visit (Visit 1) | days (Visits 2 & 3) |
| Subject Sign-in: | X | |
| Medical History | | |
| Informed Consent | | |
| form | | |
| Current oral habits | | |
| Demographics | | |
| Concomitant | | |
| Medications | | |
| Diary Card | | |
| Questionnaire | | |
| Continuance Criteria | X | X |
| Oral Soft/Hard Tissue | X | X |
| (OSHT) evaluation | | |
| VMI assessment | X | X |
| Digital Photos of | X | X |
| anterior mandibular | | |
| lingual teeth | | |
| Randomization | X | |
| Supervised product use | X^{a} | |
| AEs | X | X |
| Compliance check | X | X |

### Identity of Investigational Products

The products used during this study are listed as follows:
1. ADA reference soft manual toothbrush: (Colgate® Classic [Pokey], Colgate-Palmolive Co., New York, NY) used with ADA Toothpaste.
2. ADA reference soft manual toothbrush: used with Formulation only.
3. Enzyme formulation: Formulation II, as provided herein.
4. ADA Accepted Standard Fluoride Toothpaste: Crest® Cavity Protection Cool Mint Gel, 0.243% Sodium Fluoride, Procter & Gamble, Cincinnati, OH 45202.

### Calculus Measurement

1. Volpe-Manhold Index (V-MI) was calculated on lingual surfaces of 6 lower anterior teeth (see FIG. 2). The Volpe-Manhold Index (V-MI)² measures calculus present on the lingual surfaces of the lower six anterior teeth. The instrument used was a standard periodontal probe, graduated in millimeters. After drying the teeth with a stream of air, the instrument was placed on the most inferior border of visible calculus and measurements are obtained in the following three planes:
   (a) Bisecting the center of the lingual surface;
   (b) Diagonally through the mesial-incisal point angle of the tooth through the area of greatest calculus height; and
   (c) Diagonally through the distal point angle of the tooth through the area of greatest calculus height.

The Examiner then assigned a score to each measurement plane. Measurements were made in 0.5 mm increments starting at 0.5. A score of zero (0) denotes that there was no calculus present at a measurable site. The V-MI was calculated for each subject by summing the millimeter scores over all sites graded. Digital photos of the lingual surfaces of 6 lower anterior teeth were taken.

### Methodology and Study Design

The study was conducted as a randomized, single-blind, single-center, parallel group, three treatment clinical trial, aimed to evaluate the safety and efficacy of enzyme formulation to remove calculus accumulations. Subjects were subjected to a Volpe-Manhold Index (V-MI) calculus examination at each visit as provided below.

Forty (40) subjects who had at least 9 mm of calculus on the lingual surface of the mandibular anterior teeth were qualified to continue participation in the study, according to the eligibility criteria. Subjects were randomly assigned to one of 3 groups (20 subjects in the control, and 10 subjects in each test group).

All treatment groups brushed with an ADA-accepted toothbrush and dentifrice for 2 minutes twice daily. The three groups followed the following regimen:

Test Group A: Brushed for two minutes with the control toothbrush and Crest twice daily.

Test Group B: Brushed for 1 minute using a toothbrush with half the amount of enzyme formulation in the syringe (1 ml/syringe). Subjects were instructed to start brushing on the lingual (tongue) side of the lower front teeth and end on those same teeth with the separate toothbrush. Subjects then brushed using the control toothpaste, expectorated, reapplied the amount remaining in the syringe, down to empty the syringe with the enzyme formulating and brushed for additional minute again starting on the tongue side of the front lower teeth and ending there after 1 minute of brushing, followed by spit, and no rinsing. Brushing with enzyme formulation was done for a minimum of 30 minutes prior to the control toothbrushing. Subjects were asked to expectorate but not to rinse after using enzyme formulation for at least 30 minutes. Subjects then brushed for two minutes with the control toothbrush and Crest twice daily. In total, the subjects brushed their teeth four times daily, twice with enzyme formulation and twice with Crest. The control toothbrush was rinsed with hot water daily to clean.

Test Group C: Prior to using the custom tray, subjects brushed for 1 minute by applying half of the syringe (1 ml/syringe) using a separate toothbrush designated for enzyme formulation only, followed by spitting and no rinsing. A custom-made tray was filled with enzyme formulation from a second syringe and the tray was worn for 30 minutes once daily. The tray was removed, the subject then brushed for one minute with the remaining enzyme formulation from the first syringe, expectorated, without rinsing for a minimum of 30 minutes. Subjects completed the oral care routine by brushing with the control toothbrush and toothpaste (Crest) for two minutes twice daily. The control toothbrush was rinsed with hot water daily to clean.

Subjects were stratified according to calculus levels and gender obtained during the screening/baseline examination. Subjects brushed twice daily, unsupervised, during a 4-week test period, returning at 2-weeks and 4-weeks for safety and V-MI examinations.

### Assessment and Data Collection

The study test phase included a total of 56 treatment sessions and 3 clinical visits over a period of 4 weeks. For each subject, assessment data was collected at baseline, 2 and 4 weeks. The average data sets were calculated for each group.

### Study Endpoints and Efficacy Assessment:

The following endpoints were set for the study:

### Primary Endpoints:

1. A significant calculus reduction in the test groups as compared to the control group following 4-weeks of treatment.
2. A significant calculus reduction in the test groups as compared to longitudinal intra-group values.
3. Safety assessment based on adverse events in either test group following 4 weeks of treatment.
4. A decrease in calculus by removal of existing deposits and/or prevention of new deposits.

### Secondary Endpoints:

1. Prevention of calculus accumulation in the treatment group, as seen by no significant increase in calculus score as compared to baseline.
2. A significant calculus reduction in the test groups as compared to the control group following 2-weeks of treatment.

### Exploratory End Points

1. Any difference in patient's mouthfeel when they rub their tongue on the back of their front teeth?
2. Any improvement in patient's breath?
3. Any change in teeth appearance or color

### Analysis

An analysis set was conducted, including all subjects using the experimental gel at least a single time. A Performance analysis set consisted of all subjects providing at least one post treatment performance measurement. Only observed data was used; missing data was not imputed.

### Subject Disposition

The safety analysis set was used for describing subject disposition.

Subject disposition was tabulated; the number of enrolled, exposed, prematurely terminated and completed subjects were summarized. A list of dropouts/terminations was prepared including reason for discontinuation, and time of discontinuation.

### Safety Analyses

Safety analyses was descriptive in nature, with SAE's and AEs tabulated by body system, preferred term, severity and relation to investigational gel. Where relevant, events were related to remote log file data. In addition, safety complaints, as described by the subjects were described and investigated.

Oral cavity examination was done according to OSHT—Oral Soft & Hard Tissue Assessment. Assessment of the oral soft and hard tissue was conducted via a visual examination of the oral cavity and perioral area utilizing a standard dental light, dental mirror, and gauze. The structures examined included the gingiva (free and attached), hard and soft palate, oropharynx/uvula, buccal mucosa, tongue, floor of the mouth, labial mucosa, mucobuccal/mucolabial folds, lips, teeth, and peroral area. All abnormal findings which had the potential to be product-related and were noted after product assignment, which were not documented at baseline, or were present at baseline but had worsened during product usage, were recorded on the AE CRF.

### Performance Analyses

Efficacy was determined by reduction of dental calculus within treatment groups (longitudinal analysis) and between groups as compared to the control group following 4 weeks of treatment. Additionally, efficacy was determined by the prevention of calculus accumulation in the treatment group, as seen by no significant increase in calculus score as compared to baseline.

The primary outcome was calculated using the Volpe-Manhold Index, and all other variables were considered secondary. The calculus scores for this index were summed to provide a total score per mouth at each clinical examination. While the analyses were performed at other measurement time points during the study, the primary timepoint was at the conclusion of the trial after 4 weeks of product use.

Statistical significance of mean data for age and scoring index was determined parametrically by analysis of variance for testing of differences between the product groups. Intergroup comparisons for each parameter were made by means of multiple range tests with compensation for gender, if necessary. Data from all subjects who completed the final assessment were used in the analysis.

The data for each scoring index was also analyzed by analysis of covariance using the baseline data as the covariate. The covariate (baseline data) was included in the statistics model for increased precision in determining the effect of the test products on the scores. The adjusted means generated by this procedure compensated for any variations between treatment groups that existed in the baseline data. This reduced variability and increased power, and also adjusted for imbalances at baseline due to subject attrition.

Longitudinal (i.e., within-treatment) comparisons were performed for VMI means using a one-sample t-test on the changes from baseline to the final examination.

All comparisons were tested at an overall 0.05 level of significance using 2-sided tests. Summary statistics were provided at all timepoints. Safety data was summarized. Adverse events experienced by all subjects receiving test products were included in the safety analyses. All cases were checked for compliance with the study protocol, and for accuracy and completeness. Those cases which did not meet these requirements were excluded from primary analyses.

Post-power analyses for those endpoints that were determined to not be statistically significant were undertaken. Thus, sample sizes needed for a properly powered study were determined.

All adverse events reported were listed, documenting course, outcome, severity, and possible relationship to study products. Comparisons among the treatment groups were made by tabulating the frequency of subjects with one or more adverse events during the trial. If required, likelihood ratio chi square tests were used to compare the frequency of subjects experiencing adverse events among treatment groups.

### Results

### Study Population/Enrollment:

The final list of subject enrollments is provided in Table 4:

TABLE 4
Enrollment and withdrawal data

| Subjects | # |
|---|---|
| Recruited/Scheduled | 44 |
| Cancelled/No show | 0 |
| Subjects Consented | 44 |
| Screening/Baseline | 4 |
| Failures | |
| Randomized to Product | 40 |
| Received Product | 40 |
| Disqualified/Dropped | 2 |
| Completed | 38 |
| Subject | 2 |
| Withdrawal/Disqualification | |

All subjects who completed the study showed satisfactory compliance. Forty subjects were randomized and thirty-eight completed the study. Among the randomized subjects 22 were females and 18 were males. The mean age was 50.4 with the minimum age being 23 years and the maximum being 74 years. Calculus baseline scores were comparable for all treatments.

### Oral Tissue Exam

The examination of the oral cavity included the gingival (free and attached), hard and soft palate, oropharynx, buccal mucosa, tongue, floor of the mouth, labial mucosa, mucobuccal/mucolabial folds, lips and perioral area was undertaken. There were no observed and/or reported evidence of any hard or soft tissue damage associated with the use of test product. A summary of the adverse events seen during examination or self-reported by the subject are provided in Table 5.

TABLE 5
Adverse events summary

| | | Examiner | | | | Resolved | |
|---|---|---|---|---|---|---|---|
| | | or Self | | AE | Related to | within | Resolved |
| # | Group | Report | AE | Rating | product | 5 days | on treatment |
| 1019 | A | Examiner | Abrasion | Mild | Likely caused | Yes | Yes |
| | | | (toothbrush) | | by | | |
| | | | on gingival | | toothbrushing | | |
| | | | margin #26 | | | | |
| 1024 | B | Examiner | Erythema | Mild | Likely caused | Yes | Yes |
| | | | on gingival | | by | | |
| | | | margin #27 | | toothbrushing | | |
| 1032 | B | Examiner | Erythema | Mild | Likely caused | Yes | Yes |
| | | | on gingival | | by | | |
| | | | margin #24 | | toothbrushing | | |
| | | | & 28 | | | | |
| 1044 | B | Examiner | Inflamed | Mild | Possibly | Yes | No |
| | | | papilla on | | | | |
| | | | anterior of | | | | |
| | | | tongue | | | | |
| 1013 | C | Examiner | Erythema | Moderate | Likely caused | Yes | Yes |
| | | | on gingival | | by | | |
| | | | margin | | toothbrushing | | |
| | | | #23-26 | | | | |
| 1044 | | Self | Burning | NA | Possibly | Yes | No |
| | | Report | tongue | | | | |
| 1012 | C | Self | Gum | NA | Possibly | Yes | Yes |
| | | Report | Sensitivity | | | | |
| 1012 | C | Self | Lip | NA | Possibly | Yes | Yes |
| | | Report | Sensitivity | | | | |

All adverse events resolved within the 5-day re-evaluation period. All adverse events (examiner and self-Reported) observed in test groups B & C were attributed to the test product. The Erythema was most likely caused by the 4 toothbrushing procedures each day compared to twice daily toothbrushing in the control group. The adverse event for Subject 1019 was toothbrush abrasion.

### Clinical Study Results

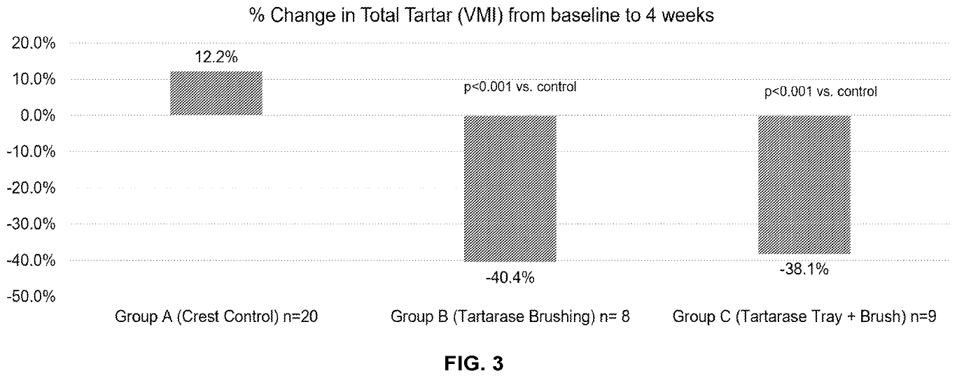

A summary of data for the Volpe-Manhold Index scores and change from baseline to Week 4 for each of the three treatments is provided in Table 6 and Table 7 and FIG. 3. Both treatments with enzyme formulation reduced calculus over the 4-week study. Treatment with enzyme formulation alone reduced calculus by 40.0% and the treatment with enzyme formulation plus custom tray with the enzyme formulation reduced calculus by 38.1% over the 4-week study. The longitudinal intra-group analysis results indicated that the two groups treated with enzyme formulation groups had statistically significant calculus reductions over the 4-week study (p<0.001). The reductions for both groups were comparable. In comparison, the control group showed increase in calculus by a statistically significant 12.2% over the 4-week study (p<0.001). The 2-week reduction for the group treated with enzyme formulation group was 0.8% and 1.6% for the group treated with enzyme formulation plus the custom tray.

TABLE 6
Volpe-Manhold index scores

| | | | | | | | | |
|---|---|---|---|---|---|---|---|---|
| Variable | n | mean | std | stderr | median | min | max | range |

Tatarase Group (B)

| | | | | | | | | |
|---|---|---|---|---|---|---|---|---|
| Baseline | 8 | 16.6 | 7.89 | 2.79 | 13.5 | 11.5 | 35.0 | 23.5 |
| Week 2 | 8 | 16.4 | 7.95 | 2.81 | 13.3 | 11.5 | 35.0 | 23.5 |
| Week 4 | 8 | 9.9 | 8.44 | 2.98 | 6.8 | 3.5 | 29.5 | 26.0 |

Tartarase Plus Custom Tray Group (C)

| | | | | | | | | |
|---|---|---|---|---|---|---|---|---|
| Baseline | 9 | 17.5 | 10.16 | 3.39 | 13.0 | 9.0 | 39.0 | 30.0 |
| Week 2 | 9 | 17.2 | 10.32 | 3.44 | 12.5 | 8.0 | 39.0 | 31.0 |
| Week 4 | 9 | 10.8 | 10.09 | 3.36 | 5.5 | 2.5 | 28.0 | 25.5 |

Control Group (A)

| | | | | | | | | |
|---|---|---|---|---|---|---|---|---|
| Baseline | 20 | 17.3 | 5.33 | 1.19 | 17.0 | 10.0 | 26.5 | 16.5 |
| Week 2 | 20 | 17.4 | 5.19 | 1.16 | 17.0 | 10.0 | 26.5 | 16.5 |
| Week 4 | 20 | 19.4 | 5.62 | 1.26 | 18.5 | 11.0 | 29.0 | 18.0 |

TABLE 7
Changes in Volpe-Manhold Index Scores over 4 weeks

| | VMI Scores & Change (%) at Week 4 | | | |
|---|---|---|---|---|
| | Baseline | Week 2 | Week 4 | Change at |
| | VMI | VMI | VM1 | Week 4 |
| Group A | 17.3 | 17.4 | 19.4 | 12.2% increase |
| Group B | 16.6 | 16.4 | 9.9 | 40.4% reduction |
| Group C | 17.5 | 17.2 | 10.8 | 38.1% reduction |

Table 8 indicates that the two treatments with enzyme formulation showed statistically superior calculus reduction compared to the Control group (p<0.001).

TABLE 8
ANCOVA for VM-I Calculus Scores at Week 4

| | | LSMean | LSMean | |
|---|---|---|---|---|
| | LSMean | Difference | Difference | |
| Contrast | Difference | Lower Cl | Upper Cl | p-value |
| Group A | 8.8 | 7.0 | 10.5 | <0.001 |
| minus | | | | |
| Group C | | | | |
| Group A | 8.8 | 7.0 | 10.6 | <0.001 |
| minus | | | | |
| Group B | | | | |
| Group C | 0.03 | −2.1 | 2.1 | 0.977 |
| minus | | | | |
| Group B | | | | |

LSMean Difference = Mean difference of the covariate adjusted treatment means
p-value = p-value of the test of the LSMean difference
LSMean Difference Lower/Upper Cl = 95% Confidence Limit Upper/Lower Bound

Table 9 provides the week 4 analysis of the covariance results. As shown in Table 7, neither of the two groups with active enzyme formulation treatment showed clinically as well as statistically significant reductions at Week 2. The confidence intervals provided in Table 8 indicates that the groups with dental treatments were significantly superior in calculus reduction compared to the control group.

TABLE 9
Week 4 Analysis of Covariance Results for Volpe-Manhold Index

| | | Std | | Pr > | | | |
|---|---|---|---|---|---|---|---|
| Effect | Estimate | error | T value | \|t\| | Alpha | Lower | Upper |
| Group A | 8.7642 | 0.8490 | 10.32 | <.0001 | 0.05 | 7.0370 | 10.4914 |
| minus | | | | | | | |
| Group C | | | | | | | |
| Group A | 8.7942 | 0.8854 | 9.93 | <.0001 | 0.05 | 6.9929 | 10.5955 |
| minus | | | | | | | |
| Group B | | | | | | | |
| Group B | 0.0300 | 1.0287 | 0.03 | 0.9769 | 0.05 | −2.0629 | 2.1229 |
| minus | | | | | | | |
| Group C | | | | | | | |

### Safety (Week 2 and Week 4)

The oral soft and hard tissue examination (OSHT) was conducted at baseline, week 2 and week 4. All findings at week 2 and week 4 were continuations of pre-conditions noted at baseline with the exception of the 4 participants (#1019, #1024, #1032, #1013) as shown in Table 5.

### Example 7: Summary of Results

The results from the study show that the use of disclosed enzyme formulation was well tolerated by study participants.

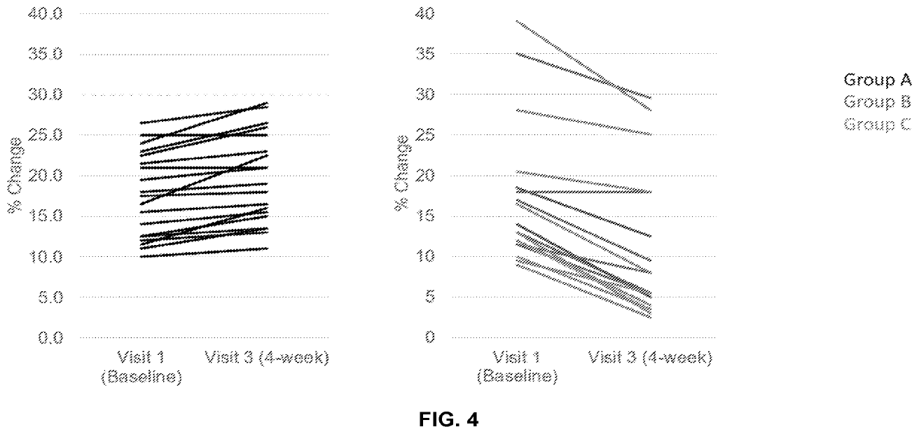

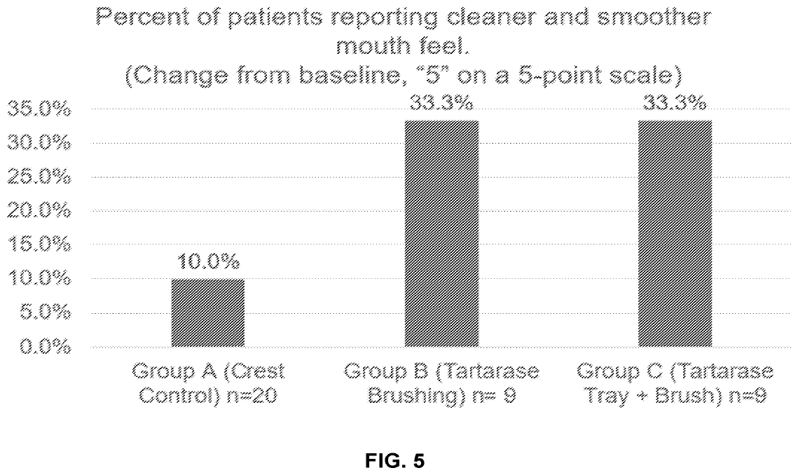

The study participants showed clinically meaningful and statistically significant reductions of calculus over the 4-week study (FIG. 4). Further, the participants in the active treatment groups gave a more favorable response to how clean their teeth felt. The positive endorsements were given by 75% of the Group B (enzyme formulation) and 67% of the Group C (enzyme formulation plus tray) compared to the control group, participants of which only gave a 50% endorsement (data not shown). Additionally, the groups B and C reported that their teeth felt cleaner, smoother, and less gritty (FIG. 5).

### Example 8: Analysis of Dental Tartar/Calculus Composition and Structure

Calculus, harvested from teeth using metal alloy dental picks was placed in 0.5 mL of water in 1.5 mL microfuge tubes. The calculus was pulverized using a Teflon pestle fitted to the microfuge tube. The contents were centrifuged, the supernatant aspirated and re-suspended in 1 mL water, and 100 microliter aliquots were drawn and placed in one or more microfuge tubes. The contents of each tube were centrifuged, supernatant aspirated and then re-suspended in 300 microliters of water in a tube containing one of the following:
Nothing added (Water control)
Trypsin
Chymotrypsin
DNase 1
Nucleanase
“Genius Nuclease”

The contents of each tube were mixed and incubated for 30 minutes at about 37° C. The contents were centrifuged, and the supernatant removed and analyzed for the presence and amount of protein, DNA and calcium. Raw quantifications were multiplied by a dilution factor (e.g., 1:3 for calcium, 1:5 for DNA, or 1:5 for protein) to achieve a total amount of protein, DNA or calcium isolated in the sample. Table 11 shows results obtained. The results shown in Table 11 indicate that Chymotrypsin and DNAase 1 were more effective than Trypsin or the other nucleases in extracting protein, DNA or calcium from calculus.

TABLE 11
Percentages of perceived maximum product yields

| | Protein (% of | DNA (% of | Calcium (% of |
|---|---|---|---|
| | control) | control) | control) |
| Control | 100 | 100 | 100 |
| Trypsin | 397 | 691.7 | 241.6 |
| Chymotrypsin | 7225.2 | 1125 | 550 |
| DNAase 1 | 3137.8 | 900 | 258.3 |
| Nucleanase | 557.4 | 300 | 50 |
| “Genius Nuclease” | 173.8 | 0 | 25 |

### Example 9: Human Clinical Investigation

### Methods

Proof of Principle human clinical investigation was conducted as a parallel group, examiner-blind, randomized, three treatment clinical trial to evaluate the safety and efficacy of a novel enzyme formulation (i.e., Tartarase) to remove existing calculus deposits in one month measured using the Volpe-Manhold Index (V-MI) on lingual surfaces of 6 lower anterior teeth. Among the secondary objectives were an evaluation of safety, assessed by examination of the gingival tissues (free and attached), hard and soft palate, oropharynx, buccal mucosa, tongue, floor of the mouth, labial mucosa, mucobuccal/mucolabial folds, lips and perioral area. In addition, a questionnaire was selected using a five-point subjective scale to evaluate oral cleanliness (1=least clean and 5=cleanest) and was included as an exploratory endpoint. Safety assessments and oral cavity examination were conducted at all visits. Forty subject consented and were enrolled with 20 randomized to the control dentifrice (Crest Cavity Protection), and 20 subjects were assigned to the Tartarase treatment groups.

The research study was approved by the U.S.IRB (U.S.IRB2022SR1/10) in accordance with ICH Guidelines E6 and the USA Food and Drug Administration and conducted by Salus Research, Inc., (an American Dental Association (ADA) Qualified Independent Research Site). The investigational product did not contain fluoride and the IRB requested all participants brush twice daily with a fluoride containing toothpaste.

Adult subjects with six (6) mandibular anterior teeth were recruited from the Salus Research database as known rapid tartar formers who measured at least 9 mm of dental calculus on the mandibular anterior six teeth, assessed using the V-MI (dental cleaning was completed between 2-6 months prior to the study initiation). Subjects who did not brush regularly, used chlorhexidine mouth rinse or required antibiotic pre-medication prior to dental procedures were excluded. Subjects were withdrawn from the study if they did not comply with product usage instructions or used any oral hygiene products other than the assigned study products. Use of dental floss, electric toothbrushes, and mouth rinses were prohibited.

During the initial visit, participants were assessed to ensure at least 9 mm of dental calculus was present on the lingual surfaces of the six mandibular anterior teeth and eligible subjects were evaluated for demographics, medical history and concomitant medications. The 40 subjects who qualified were randomly assigned to one of the three study groups by using a randomization schedule developed and maintained by an independent statistician. Subjects were stratified by baseline lingual V-MI dental calculus scores and gender. Half of the test subjects (n=20) were randomized to the control group and 10 to each of the Tartarase arms (Tartarase Brush only and Tartarase Brush+tray). All subjects performed their first use supervised at the research site then unsupervised during the 4-week test. For each subject, assessment data was collected at baseline, 2 and 4 weeks (Table 12). All test subjects brushed twice daily with the control toothpaste Crest® Cavity Protection Cool Mint Gel, 0.243% Sodium Fluoride using the same an ADA reference soft manual toothbrush. Test group assignments were as follows:
1. Control (20 subjects): Brush twice daily for two minutes with ADA toothbrush and Crest.
2. Tartarase Brush (10 subjects): Brush twice daily with Tartarase and twice with Crest.
3. Tartarase Tray (10 subjects): A custom dental tray was made for all Tartarase Tray subjects. Test subjects brushed 1 minute with Tartarase then applied Tartarase using the custom tray (worn once daily for 30 minutes). This procedure was followed by another brushing with Tartarase for 1 minute. The Tartarase brush/tray/brush was performed once daily. Participants also brushed twice daily with Crest.

TABLE 12
Baseline characteristics, including the mean Volpe-Manhold Index (V-MI)of the
different treatment groups, demographic data of the different treatment groups
and the normal brushing habits of those in the different treatment groups.

| | Crest | | Tartarase | | Tartarase | | | |
|---|---|---|---|---|---|---|---|---|
| | Control | | Brushing | | Tray | | Total | |
| Randomized | 20 | | 10 | | 10 | | 40 | |
| Completed | 20 | | 8 | | 9 | | 37 | |
| Mean VMI at | 17.3 | | 16.6 | | 17.5 | | | |
| Baseline | | | | | | | | |
| Mean Age in | 50.2 | | 58.9 | | 42.3 | | 50.4 | (p = 0.234) |
| Years | | | | | | | | |
| Gender | | | | | | | | |
| Male | 9 | (45.0%) | 5 | (50.0%) | 4 | (40.0%) | 18 | (45.0%) |
| Female | 11 | (55.0%) | 5 | (50.0%) | 6 | (60.0%) | 22 | (55.0%) |
| Race/Ethnicity | | | | | | | | |
| White/ | 15 | (75.0%) | 10 | (100.0%) | 8 | (80.0%) | 33 | (82.5%) |
| Caucasian | | | | | | | | |
| Black/African | 3 | (15.0%) | | | 2 | (20.0%) | 5 | (12.5%) |
| Heritage | | | | | | | | |
| Multiracial | 2 | (10.0%) | | | | | 2 | (5.0%) |
| Mean times brush | 1.8 | | 1.9 | | 1.6 | | 1.7 | |
| teeth daily | | | | | | | | |

### Tartarase Formulation

The formulation was prepared in two batches, each sufficient for 2.0 ml/day for 14 days for 20 participants. Ceteareth-25 (0.23%), glycerol (13.6%), Xanthan gum (0.28%), sorbitol (4.4%) and hydrated silica (11%). DNase 1 (197, 326 Kunitz units/ml), and chymotrypsin (3,333 units/ml). The enzymes were obtained from Worthington Biochemicals.

For this 1-month study, the test product was formulated using twice the levels per unit volume previously shown to be efficacious in preclinical studies using canine calculus.

### Efficacy Assessments and Statistical Analysis

Efficacy was determined by the change is dental calculus within treatment groups (longitudinal analysis) and between groups as compared to the control group following 4 weeks of treatment. Additionally, efficacy in the Tartarase treatment groups was determined by the prevention of calculus accumulation, assessed by no significant increase in calculus score as compared to baseline. The primary outcome was calculus abundance using the Volpe-Manhold Index, and all other variables are considered secondary. The measurement is performed on the lingual surface of the 6 mandibular anterior teeth. The calculus scores for this index were summed to provide a total score per mouth at each clinical examination. The primary timepoint was at the conclusion of the trial after 4 weeks of product use.

Data from all subjects who completed the final assessment were used in the analysis. The data for each scoring index was also analyzed by analysis of covariance using the baseline data as the covariate. The covariate (baseline data) was included in the statistics model for increased precision in determining the effect of the test products on the scores. The adjusted means generated by this procedure compensate for any variations between treatment groups that existed in the baseline data. This reduces variability and increases power, and also adjusts for imbalances at baseline due to subject attrition. Longitudinal (i.e., within-treatment) comparisons were performed for V-MI means using a one-sample t-test on the changes from baseline to the final examination. All comparisons were tested at an overall 0.05 level of significance using 2-sided tests.

### Safety Assessments

An oral cavity examination was conducted at all visits including the gingival (free and attached), hard and soft palate, oropharynx, buccal mucosa, tongue, floor of the mouth, labial mucosa, mucobuccal/mucolabial folds, lips and perioral area. In addition, subjects were asked if they had an any adverse events (AEs) since the prior visit. AEs were classified as serious or non-serious and by severity, as anticipated or unanticipated, and possible relationship to the test product. All AEs (examiner and self-report) were evaluated on subsequent visits to determine if AEs had resolved.

### Results

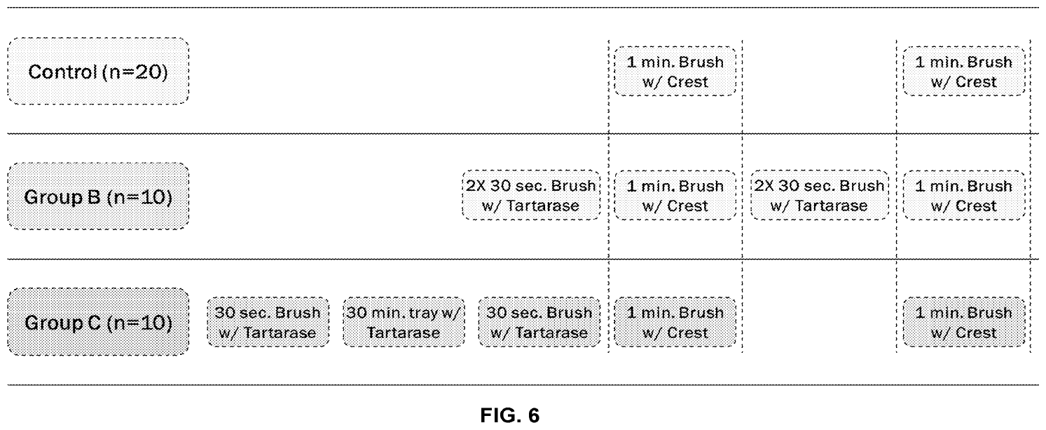

Overview of daily brushing schedule by study arm is shown in FIG. 6. Forty (40) subjects were randomized and thirty-seven (37) completed the study. One patient was unwilling to comply with study procedures and the investigator removed the patient from the study and two patients withdrew consent. For randomized subjects, the gender distribution was 22 females and 18 males. The mean age was 50.4 years with the minimum age being 23 years and the maximum being 74 years. Caucasians represented 33 of the 40 subjects (82.5%) and none of the patients self-identified as Hispanic or Latino. Calculus baseline scores were comparable for all treatments. Subjects reported similar daily oral hygiene routines (twice daily).

### Efficacy: Dental Calculus

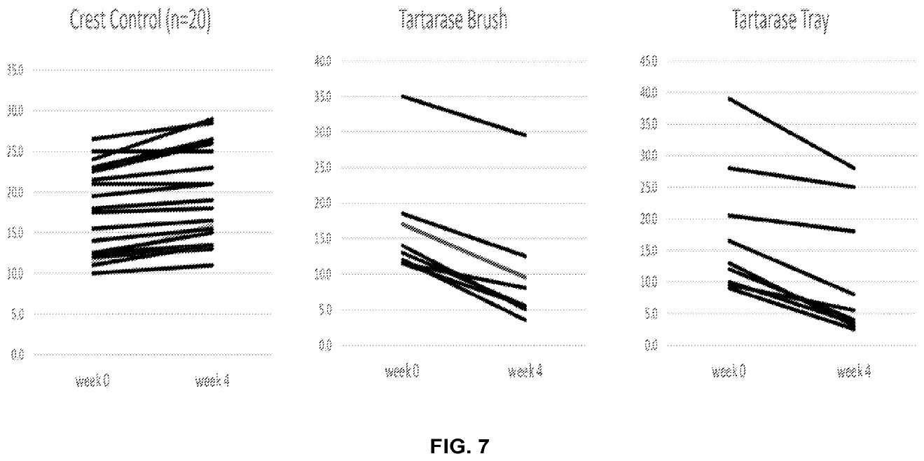

Both treatments with Tartarase reduced calculus over the 4-week study. Specifically, the Tartarase Brushing only group removed calculus by 40.0% and the Tartarase Brushing+Tray by 38.1%. The longitudinal intra group analysis indicated that the two Tartarase groups had statistically significant calculus reductions over the 4-week study (p<0.001) and were statistically different from the Crest control group (Table 13). The results for both Tartarase treatment groups were comparable and all 17 assigned to the experimental product demonstrated a reduction in calculus (FIG. 7). In comparison, the Crest control group resulted in a statistically significant 12.2% increase in calculus over the 4-week study (p<0.001). Moreover, the test subjects in the Crest control group had an increase in calculus deposition (FIG. 7). None of the test groups had a significant change at the 2-week examination evaluation.

TABLE 13
Mean baseline and 4-week V-MI measures and percent change and the
direction of change in the V-MI of the different treatment groups

| | Baseline | Week 4 | Change to |
|---|---|---|---|
| | V-MI | V-MI | Week 4 |
| Tartarase | 16.6 | 9.9 | 40.4% reduction |
| Brushing | | | |
| Tartarase Tray | 17.5 | 10.8 | 38.1% reduction |
| Crest Control | 17.3 | 19.4 | 12.2% increase |

### Oral Cleanliness Questionnaire

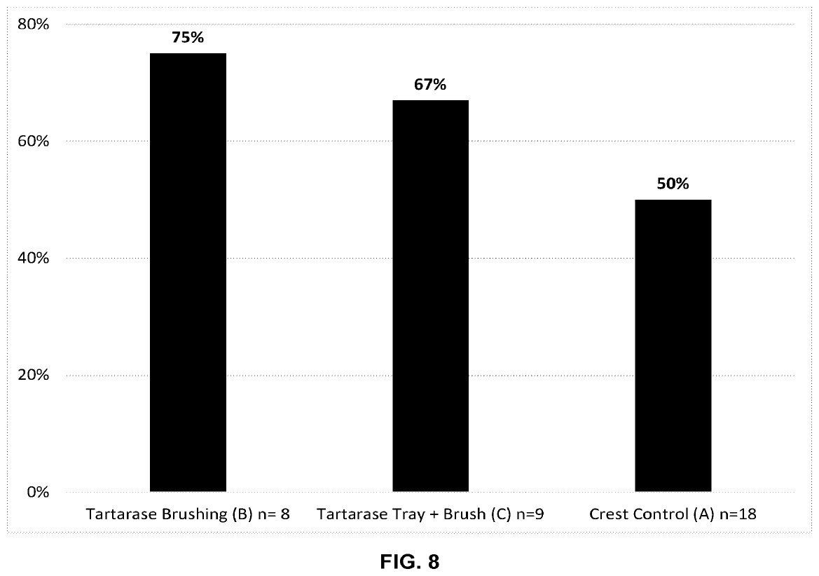

The Tartarase treatment group participants reported a more favorable response to how clean their teeth felt compared to the control group. The positive endorsements of ‘4’ or ‘5’ (5=cleanest) were self-reported by 75% of the Tartarase Brush only and 67% of the Tartarase Brush+Tray group (FIG. 8). The Control group reported a 50% positive endorsement to the two categories of ‘4’ or ‘5’. The test subjects were provided an opportunity to offer their overall impressions of the Tartarase product they were assigned on oral cleanliness including:
1. “I can feel each tooth in my mouth, and they feel smooth.”
2. “I can feel my individual teeth better, my teeth—inside bottom teeth feel cleaner.”
3. “I feel like my mouth stays clean and fresh throughout the day. My breath smells better.”

### Safety Issues

No safety issues were noted from the use of Tartarase and there were no serious AEs nor any SAEs. Tartarase was well tolerated. There were 5 AEs based upon oral examination (4 AEs were attributed to toothbrush abrasion and the other was an inflamed papilla (mild) on anterior of the tongue). In total, the control group had 1 AE, there were 3 AEs in the Tartarase brushing group and 1 AE in the Tartarase Tray group (Table 14). There were no oral hard tissue changes noted during the 4-week trial. Three test subjects self-reported AEs with 2 who listed gum or lip sensitivity (Tartarase tray group) and 1 experienced a burning tongue (Tartarase Brushing group). All AEs (Examiner observation and self-reported) resolved within the 5-day re-evaluation period (Table 14).

TABLE 14
Summary of adverse events

| | | | | | | | Resolved |
|---|---|---|---|---|---|---|---|
| | | Reporting | | AE | Related to | Resolved | within |
| Group | Subject | Entity | AE | Rating | product? | on treatment | 5 days |
| A | 1019 | Examiner | Abrasion | Mild | No, likely | Yes | Yes |
| | | | on | | caused by | | |
| | | | gingival | | toothbrushing | | |
| | | | margin | | | | |
| B | 1024 | Examiner | Erythema | Mild | No, likely | Yes | Yes |
| (Brush) | | | on | | caused by | | |
| | | | gingival | | toothbrushing | | |
| | | | margin | | | | |
| | 1032 | Examiner | Erythema | Mild | No, likely | Yes | Yes |
| | | | on | | caused by | | |
| | | | gingival | | toothbrushing | | |
| | | | margin | | | | |
| | 1044 | Examiner | Inflamed | Mild | Possibly | No | Yes |
| | | | papilla on | | | | |
| | | | anterior | | | | |
| | | | of tongue | | | | |
| | | Patient | Burning | NA | Possibly | No | Yes |
| | | | tongue | | | | |
| C | 1013 | Examiner | Erythema | Moderate | No, likely | Yes | Yes |
| (Brush + tray) | | | on | | caused by | | |
| | | | gingival | | toothbrushing | | |
| | | | margin | | | | |
| | 1012 | Patient | Gum | NA | Possibly | Yes | Yes |
| | | | Sensitivity | | | | |
| | | Patient | Lip | NA | Possibly | Yes | Yes |
| | | | Sensitivity | | | | |

### Summary of Example 9

Tartarase significantly reduced dental calculus after 4 weeks with approximately a 40% reduction in both Tartarase test groups while the Crest Control group demonstrated a 12% increase of dental calculus deposits. Moreover, every Tartarase test subject had a decrease in dental calculus after 4-weeks of daily, unsupervised use. Without wishing to be bound by theory, the results of this study support the theory that DNA and proteins together provide structural support of dental calculus, and that enzymes target DNA and denature proteins can be especially effective in disrupting the structure of dental calculus and thereby reversing dental calculus build-up. Equally important are the results showing evidence of no further tartar build up, indicating that the Tartarase formula is effective in preventing tartar formation following a dental cleaning. Tartarase test subjects demonstrated an approximate 40% calculus reduction in 4 weeks while the control group experienced a 12% increase in calculus. Fabrication of a custom tray for application is contemplated.

As the active ingredients of Tartarase are enzymes, a general feature of enzymes is important to consider. Enzymes are protein catalysts. They facilitate a chemical reaction that would normally occur to happen at an enhanced velocity. In addition, they allow the reaction to occur in both forward and reverse directions. Thus, with these types of enzymes the accumulating products inhibit the enzymes from catalyzing their forward reactions. This could be a reason why the Tartarase tray group did not out-perform the Tartarase brushing group. In addition, the in-vitro studies have found that the enzymes act extremely quickly, with the reactions completed in <5 minutes, indicating that the 30-minute incubation time in the tray group provided no advantage.

In the pre-clinical studies, data supported the importance of a two-enzyme formulation. It was speculated that the only way calculus can be attacked was at its outer 2-dimensional surface. The model of calculus structure was of a calcified complex of fundamental biopolymers. If an enzyme that has specificity for only one of the complex biopolymers, then when the target biopolymers has been consumed the enzyme can go no further, as the remaining target is concealed by biopolymers that are not targets for the single enzyme. Thus, using the enzymes in combination allows for a progressive mechanism that systematically peals away calculus from its outermost surface, eventually reaching the tooth enamel.

The clinical study has demonstrated a capacity to reduce calculus. The Tartarase toothpaste tested demonstrated a significant calculus removal in a 4-week trial period with test subjects who were known calculus-formers that had their teeth cleaned between 2 and 6 months prior to entering the research study. The trial suggested that the Tartarase formula has potential to reduce calculus when used in a daily routine of oral hygiene.

This experiment was designed to evaluate the safety and efficacy of a novel enzyme formulation (i.e., Tartarase) to remove existing calculus deposits in one month, measured using the Volpe-Manhold Index (V-MI) on lingual surfaces of 6 lower anterior teeth. The test formulation, containing a large excess of the active enzymes in a common mixture of inactive ingredients, was compared to Crest Cavity Protection, as a control dentifrice. A total of 40 randomized test subjects began the study with 20 assigned to the control dentifrice and 20 assigned to the Tartarase groups (one brushing with Tartarase twice daily and one brushed with Tartarase and wore a dental tray filled with Tartarase for 30 minutes then brushed again with Tartarase). The results demonstrated a 12% calculus increase for the Crest control group compared to a 40% calculus reduction in the Tartarase groups.

TABLE 15
SEQUENCES

| SEQ | | | |
|---|---|---|---|
| ID | TYPE | SOURCE | SEQUENCE |
| 1 | Protein | Bos Taurus | MRGTRLMGLLLALAGLLQLGLSLKIAAFNI |
| | | DNAse 1 | RTFGETKMSNATLASYIVRIVRRYDIVLIQ |
| | | | EVRDSHLVAVGKLLDYLNQDDPNTYHYVVS |
| | | | EPLGRNSYKERYLFLFRPNKVSVLDTYQYD |
| | | | DGCESCGNDSFSREPAVVKFSSHSTKVKEF |
| | | | AIVALHSAPSDAVAEINSLYDVYLDVQQKW |
| | | | HLNDVMLMGDFNADCSYVTSSQWSSIRLRT |
| | | | SSTFQWLIPDSADTTATSTNCAYDRIVVAG |
| | | | SLLQSSVVPGSAAPFDFQAAYGLSNEMALA |
| | | | ISDHYPVEVTLT |
| 2 | Protein | Bos Taurus | CGVPAIQPVLSGLSRIVNGEEAVPGSWPWQ |
| | | Chymotrypsinogen | VSLQDKTGFHFCGGSLINENWVVTAAHCGV |
| | | | TTSDVVVAGEFDQGSSSEKIQKLKIAKVFK |
| | | | NSKYNSLTINNDITLLKLSTAASFSQTVSA |
| | | | VCLPSASDDFAAGTTCVTTGWGLTRYTNAN |
| | | | TPDRLQQASLPLLSNTNCKKYWGTKIKDAM |
| | | | ICAGASGVSSCMGDSGGPLVCKKNGAWTLV |
| | | | GIVSWGSSTCSTSTPGVYARVTALVNWVQQ |
| | | | TLAAN |
| 3 | Protein | Artificial | CGVPAIQPVLSGL |
| | | Chymotypsin | |
| | | chain A | |
| 4 | Protein | Artificial | SRIVNGEEAVPGSWPWQVSLQDKTGFHFCG |
| | | Chymotypsin | GSLINENWVVTAAHCGVTTSDVVVAGEFDQ |
| | | chain B | GSSSEKIQKLKIAKVFKNSKYNSLTINNDI |
| | | | TLLKLSTAASFSQTVSAVCLPSASDDFAAG |
| | | | TTCVTTGWGLTRY |
| 5 | Protein | Artificial | ANTPDRLQQASLPLLSNTNCKKYWGTKIKD |
| | | Chymotypsin | AMICAGASGVSSCMGDSGGPLVCKKNGAWT |
| | | chain C | LVGIVSWGSSTCSTSTPGVYARVTALVNWV |
| | | | QQTLAAN |
| 6 | Protein | Bos Taurus | MPGVVRLLALLLVPLLLGSARGLHNATQRT |
| | | Beta- | FQIDYRRNRFLKDGQPFRYISGSIHYFRVP |
| | | galactosidase | RFYWKDRLLKMKMAGLNAIQTYVAWNFHEL |
| | | | QPGRYNFSGDHDVEHFIQLAHELGLLVILR |
| | | | PGPYICAEWDMGGLPAWLLEKKSIVLRSSD |
| | | | PDYLAAVDKWLGVLLPKMRPLLYKNGGPII |
| | | | TVQVENEYGSYLSCDYDYLRFLQKRFHDHL |
| | | | GEDVLLFTTDGVNERLLQCGALQGLYATVD |
| | | | FSPGTNLTAAFMLQRKFEPTGPLVNSEFYT |
| | | | GWLDHWGQRHSTVSSKAVAFTLHDMLALGA |
| | | | NVNMYMFIGGTNFAYWNGANIPYQPQPTSY |
| | | | DYDAPLSEAGDLTEKYFALRDIIQKFAKVP |
| | | | EGPIPPSTPKFAYGKVALNKLKTVEDALNI |
| | | | LCPSGPIKSVYPLTFIDVKQYFGFVLYRTM |
| | | | LPEDCSDPTPLSSPLSGVHDRAYVSVNGVA |
| | | | QGILERESVITLNITGKAGATLDLLVENMG |
| | | | RVNYGSSINDFKGLVSNLTLGSKILTNWEI |
| | | | FPLDMEDAVRSHLGTWGGRDRGYHNKARAH |
| | | | SPPTYALPTFYVGNFTIPSGIADLPQDTFI |
| | | | QFPGWTKGQVWINGFNLGRYWPVRGPQMTL |
| | | | FVPQHILVTSTPNTIVVLELEHAPCQDGGP |
| | | | ELCTVEFVDKPVFRTVQTHRHAN |

What is claimed is:

1. A dental composition comprising:
a. a DNase and/or RNase;
b. chymotrypsin or a functional variant or derivative thereof; and
c. an orally acceptable additive, carrier or excipient or any combination thereof.

2. The dental composition of claim 1, wherein the composition does not comprise β-galactosidase.

3. The dental composition of any one of claim 1 or 2, wherein the DNAse is present in an amount in Kunitz units/mL of about 50,000 to about 250,000, or about 100,000 to about 200,000, or about 150,000 to about 200,000, or 150,000, or 160,000, or 170,000, or 180,000, or 185,000 or 190,000, or 200,000.

4. The dental composition of any one of claim 1-3, wherein the DNAse is DNAse I or a functional variant or derivative thereof.

5. The dental composition of any one of claims 1-4, wherein chymotrypsin or a functional variant or derivative thereof is present in functional units based on the rate of hydrolysis of a chromogenic substrate in an amount of about 100 to about 5,000, or about 500 to about 4,000, or about 1000 to about 3000, or 1,500, or 1,600, or 1,700, or 1,800, or 1,900, or 2,000, or 2,100, or 2,200, or 2,300, or 2,400, or 2,500, or 2,600, or 2,700, or 2,800, or 2,900 or 3,000 units/mL.

6. The dental composition of claim 1, wherein the orally acceptable additive is a thickener and/or a gelling agent selected from gellan gum (low acyl or high acyl), glycerol, silica, guar gum, xanthan gum, polyethylene glycols, polyvinyl pyrrolidones and co-polymers thereof, polylactic acids, polyglyocolic acids, long chain fatty acid alcohols, cellulose-based polymers, acrylate polymers and any combination thereof.

7. The dental composition of claim 1, wherein the orally acceptable carriers is selected from ethanol, isopropanol, glycerol and sorbitol.

8. The dental composition of claim 1, further comprising a surfactant selected from Ceteareth or Steareth.

9. The dental composition of claim 1, wherein the composition comprises:
a. DNase I or a functional variant or derivative thereof,
b. Chymotrypsin or a functional variant or derivative thereof;
and one or more of,
c. 10%-20% glycerol,
d. 0.000001%-0.00001% Ceteareth-25,
e. 0.1%-0.5% Xanthan gum,
f. 1%-10% sorbitol, and
g. 5-25% silica.

10. The composition of claim 9, further comprising water.

11. An oral hygiene device comprising the composition of any one of claims 1-10, wherein the composition is combined with a dental tray, a capsule applicator, an oral pick or a dental floss.

12. Use of the composition or device of any one of claims 1-11, for removing calculus dentalis in a subject in need thereof.

13. The use of the composition of claim 12, wherein the subject is a mammal.

14. The use of the composition of claim 12, wherein the subject is a human.

15. A method for reducing or removing calculus dentalis in a subject in need thereof, the method comprising contacting a tooth surface with an effective amount of a composition comprising:
a. DNAse I or a functional variant or derivatives thereof, and
b. chymotrypsin or a functional variant or derivative thereof.

16. The method of claim 15, wherein the composition does not comprise β-galactosidase.

17. The method of claim 15, wherein the composition further comprises an orally acceptable additive, carrier or excipient or a combination thereof.

18. The method of claim any one of claims 15-17, wherein the composition comprises a paste or a liquid and contacting a tooth surface comprises application of the composition using a brush, a tray, by oral rinse or any combination thereof.

19. The method of claim 18, wherein the method comprises at least once daily, at least twice daily or at least three-times daily applications for a period of at least a week.

20. A method for reducing or removing calculus dentalis in a subject in need thereof, the method comprising contacting a tooth surface with an effective amount of a composition of any one of claims 1-10.

21. The method of any one of claims 15-20, wherein the subject is a mammal.

22. The method of claim 21, wherein the subject is a canine, equine, feline or primate.

23. The method of claim 21, wherein the subject is a human.

24. A kit comprising:
a. a composition of any one of claims 1-10;
b. instructions for use.

25. The kit of claim 24, further comprising at least one applicator selected from a toothbrush, an oral pick, a dental floss, and a dental tray.


SEQUENCE LISTING (6 sequences)

SEQ ID NO: 1 - protein, 282 aa, Bos taurus
MRGTRLMGLLLALAGLLQLGLSLKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 2 - protein, 245 aa, Bos taurus
CGVPAIQPVLSGLSRIVNGEEAVPGSWPWQVSLQDKTGFHFCGGSLINENWVVTAAHCGVTTSDVVVAGEFDQGSSSEKIQKLKIAKVFKNSKYNSLTINNDITLLKLSTAASFSQTVSAVCLPSASDDFAAGTTCVTTGWGLTRYTNANTPDRLQQASLPLLSNTNCKKYWGTKIKDAMICAGASGVSSCMGDSGGPLVCKKNGAWTLVGIVSWGSSTCSTSTPGVYARVTALVNWVQQTLAAN

SEQ ID NO: 3 - protein, 13 aa, synthetic construct
CGVPAIQPVLSGL

SEQ ID NO: 4 - protein, 133 aa, synthetic construct
SRIVNGEEAVPGSWPWQVSLQDKTGFHFCGGSLINENWVVTAAHCGVTTSDVVVAGEFDQGSSSEKIQKLKIAKVFKNSKYNSLTINNDITLLKLSTAASFSQTVSAVCLPSASDDFAAGTTCVTTGWGLTRY

SEQ ID NO: 5 - protein, 97 aa, synthetic construct [sequence omitted]

SEQ ID NO: 6 - protein, 653 aa, Bos taurus
MPGVVRLLALLLVPLLLGSARGLHNATQRTFQIDYRRNRFLKDGQPFRYISGSIHYFRVPRFYWKDRLLKMKMAGLNAIQTYVAWNFHELQPGRYNFSGDHDVEHFIQLAHELGLLVILRPGPYICAEWDMGGLPAWLLEKKSIVLRSSDPDYLAAVDKWLGVLLPKMRPLLYKNGGPIITVQVENEYGSYLSCDYDYLRFLQKRFHDHLGEDVLLFTTDGVNERLLQCGALQGLYATVDFSPGTNLTAAFMLQRKFEPTGPLVNSEFYTGWLDHWGQRHSTVSSKAVAFTLHDMLALGANVNMYMFIGGTNFAYWNGANIPYQPQPTSYDYDAPLSEAGDLTEKYFALRDIIQKFAKVPEGPIPPSTPKFAYGKVALNKLKTVEDALNILCPSGPIKSVYPLTFIDVKQYFGFVLYRTMLPEDCSDPTPLSSPLSGVHDRAYVSVNGVAQGILERESVITLNITGKAGATLDLLVENMGRVNYGSSINDFKGLVSNLTLGSKILTNWEIFPLDMEDAVRSHLGTWGGRDRGYHNKARAHSPPTYALPTFYVGNFTIPSGIADLPQDTFIQFPGWTKGQVWINGFNLGRYWPVRGPQMTLFVPQHILVTSTPNTIVVLELEHAPCQDGGPELCTVEFVDKPVFRTVQTHRHAN